(19) United States
(12) Patent Application Publication
(10) Pub. No.: US 2026/0211485 A1
(43) Pub. Date: Jul. 23, 2026

(54) METHODS AND SYSTEMS FOR MODELLING TIME-VARIANT PSYCHOSENSORY ATTENTION

(71) Applicant: Musyfy Inc., Mississauga (CA)

(72) Inventor: Sarbjit S. PARHAR, Mississauga (CA)

(21) Appl. No.: 19/372,100

(22) Filed: Oct. 28, 2025

Related U.S. Application Data
(60) Provisional application No. 63/747,199, filed on Jan. 20, 2025.

(51) Int. Cl. G06F 3/01; G06N 3/004

(52) U.S. Cl. CPC G06F 3/011; G06N 3/004; G06F 2203/011

(57) ABSTRACT

Measuring or quantifying human interactions with products and services may be difficult. An example solution provides a computer-implemented method. The method includes receiving, in response to an external stimulus over a period of time, human sensory data. The human sensory data includes a plurality of time intervals. The method further includes determining an emotional response based on the human sensory data. The method further includes assigning a numeric value to the emotional response. The method further includes computing a change of the emotional response over each of the plurality of time intervals over the period of time.

### CROSS-REFERENCE TO RELATED APPLICATIONS

This application claims priority to U.S. Patent Application No. 63/747,199 filed on Jan. 20, 2025, entitled “Methods and Systems for Modelling Time-Variant Psychosensory Attention”, which is incorporated herein by reference in its entirety.

### TECHNICAL FIELD

Example embodiments relate to methods and systems for modelling time-variant psychosensory attention to determine successful human interactions.

### BACKGROUND

Many industries rely on an understanding of how consumers or users will engage with their products or services to determine the viability of these products or services. Industries may rely on several streams of product and user data to this end. In some cases, businesses may alter their product and services or market them in different manners based on perceived expectations to improve their products and services. Similar processes may occur in workplace management or administration. Processing data to arrive at firm conclusions about the merits of a product, service or dynamic may prove challenging.

Measuring or quantifying human interactions with products and services may present difficulties for several reasons. For instance, different products and services may seek to offer distinct value propositions or to target specific demographics. Furthermore, an individual's interaction with a product or service may vary depending on their specific individual characteristics and be dependent upon their expectations of and the form of the product or service with which they are interacting. In addition, sources of data are often disparate and challenging to process and manage when isolated. Consequently, many current processes can be time-consuming, costly and may find it elusive to establish firm conclusions.

Therefore, there is need for an improved method and system to quantify human interactions with products or services and determine their potential for success.

### SUMMARY

According to a first aspect, there is provided a computer-implemented method, comprising: receiving, in response to an external stimulus over a period of time, human sensory data that includes a plurality of time intervals; determining an emotional response based on the human sensory data; assigning a numeric value to the emotional response; and computing a change of the emotional response over each of the plurality of time intervals over the period of time.

In some embodiments, the external stimulus may be a form of media, comprising at least one of an advertisement, a video a film, text, an image, or audio. The method may further include predicting the financial success of the media based on at least one of the emotional response or change of the emotional response.

In some embodiments, the human sensory data may include data describing at least one of vision, hearing, touch, taste or smell.

In some embodiments, the human sensory data may be collected by biometric sensors or manual observation.

In some embodiments, a desired emotional response may be a sigmoidal curve where the emotional response is at a low value at the beginning of the period of time and rises to a higher value at the conclusion of the period of time.

In some embodiments, the human sensory data may be produced by an artificial intelligence agent.

In some embodiments, the artificial intelligence agent may be trained to emulate a user with a specific disposition.

In some embodiments, the method may further include: computing a derivative of the change in emotional response over the period of time; and determining a success metric associated with the external stimulus based on the derivative of the change of the emotional response over the period of time as well as the change of emotional response over the period of time compared with a desired emotional response.

In some embodiments, the method may further include generating a recommendation to adapt the external stimulus based on a success metric associated with the external stimulus.

According to another aspect, there is provided a system, comprising: a user interface component; and a server component configured to: receive in response to an external stimulus over a period of time, human sensory data that includes a plurality of time intervals; determine an emotional response based on the human sensory data; assign a numeric value to the emotional response; compute a change of the emotional response over each time of the plurality of time intervals over the period of time.

In some embodiments, the external stimulus may be a form of media, comprising at least one of an advertisement, a video a film, text, an image, or audio, the system further configured to predict financial success of the media based on at least one of the emotional response or change of the emotional response.

In some embodiments, the human sensory data may include data describing at least one of vision, hearing, touch, taste or smell.

In some embodiments, the human sensory data may be collected by biometric sensors or manual observation.

In some embodiments, a desired emotional response may be a sigmoidal curve where the emotional response is at a low value at the beginning of the period of time and rises to a higher value at the conclusion of the period of time.

In some embodiments, the human sensory data may be produced by an artificial intelligence agent.

In some embodiments, the artificial intelligence agent may be trained to emulate a user with a specific disposition.

In some embodiments, the server component may be further configured to: compute a derivative of the change in emotional response over the period of time; and determine a success metric associated with the external stimulus based on the derivative of the change of the emotional response over the period of time as well as the change of emotional response over the period of time compared with a desired emotional response.

In some embodiments, the server may be further configured to generate a recommendation to the external stimulus based on a success metric associated with the external stimulus.

According to another aspect, there is provided one or more non-transitory computer readable media storing executable instructions thereon that, when executed by at least one computer, cause the at least one computer to perform a method comprising: receiving, in response to an external stimulus over a period of time, human sensory data that includes a plurality of time intervals; determining an emotional response based on the human sensory data; assigning a numeric value to the emotional response; and computing a change of the emotional response over each of the plurality of time intervals over the period of time.

In some embodiments, the external stimulus may be a form of media, comprising at least one of an advertisement, a video a film, text, an image, or audio, the method further comprising predicting the financial success of the media based on at least one of the emotional response or change of the emotional response.

According to another aspect, there is provided a computer-implemented method for determining successful human interactions, the method comprising: receiving human sensory data in response to an external stimulus over a period of time that includes more than one time intervals; determining an emotional response based on the human sensory data; assigning a numeric value to the emotional response; and computing a change of the emotional response over each of the time intervals of the period of time.

In some embodiments, the method may further include: determining a success metric based on the change of the emotional response over the period of time compared with a desired emotional response.

In some embodiments, the method may further include generating a recommendation to adapt the external stimulus based on the success metric.

In some embodiments, the method may further include adapting the external stimulus based on the recommendation.

In some embodiments, the human sensory data may include a plurality of sets of data, the plurality of sets of data relating to at least one of vision, hearing, touch, taste or smell.

In some embodiments, each step of the method may be performed for each of the plurality of sets of data.

In some embodiments, the method may further include: computing a derivative of the change in emotional response over the period of time; and determining the success metric based on the derivative of the change of the emotional response over the period of time as well as the change of emotional response over the period of time compared with a desired emotional response.

In some embodiments, the human sensory data may be collected by manual observation.

In some embodiments, the human sensory data may be collected by biometric sensors.

In some embodiments, the human sensory data may be collected from an artificial intelligence agent.

In some embodiments, the human sensory data may be produced by an artificial intelligence engine.

In some embodiments, the method may further include: predicting, by the artificial intelligence agent, an emotional response to the external stimulus.

In some embodiments, the artificial intelligence agent may be trained to emulate a user with a specific disposition.

In some embodiments, the human sensory data may be produced based on a user with a specific disposition.

In some embodiments, predicting the emotional response may further include the artificial intelligence agent emulating a user with a specific disposition.

In some embodiments, the specific disposition may be at least one of age or gender.

In some embodiments, the external stimulus may be a form of media.

In some embodiments, the form of media may be an advertisement.

In some embodiments, the form of media may be at least one of a video or film.

In some embodiments, the human sensory data may be produced by the artificial intelligence engine through user engagement analysis, video content analysis or natural language processing.

In some embodiments, the desired emotional response may be a sigmoidal curve where the emotional response is at a low value at the beginning of the period of time and rises to a higher value at the conclusion of the period of time.

In some embodiments, the artificial intelligence engine may be used to predict the financial success of the media.

In some embodiments, the external stimulus may be an experience.

In some embodiments, the experience may be a process of driving a vehicle.

In another aspect, there is provided a system for determining successful human interactions, the system comprising: a user interface component; and a server component configured to: receive human sensory data in response to an external stimulus over a period of time that includes more than one time intervals; determine an emotional response based on the human sensory data; assign a numeric value to the emotional response; and compute a change of the emotional response over each time of the time intervals of the period of time.

In some embodiments, the server may be further configured to: determine a success metric based on the change of the emotional response over the period of time compared with a desired emotional response.

In some embodiments, the server may be further configured to generate a recommendation to the external stimulus based on the success metric.

In some embodiments, the server may be further configured to transmit the recommendation to the user interface component.

In some embodiments, the human sensory data may include a plurality of sets of data, the plurality of sets of data relating to at least one of vision, hearing, touch, taste or smell.

In some embodiments, the server may be further configured to: compute a derivate of the change in emotional response over the period of time; and determine the success metric based on the derivative of the change of the emotional response over the period of time as well as the change of emotional response over the period of time compared with a desired emotional response.

In some embodiments, the system may further include biometric sensors for collecting the human sensory data.

In some embodiments, the system may further include an artificial intelligence agent.

In some embodiments, the artificial intelligence agent may be further configured to collect the human sensory data.

In some embodiments, the system may further include an artificial intelligence engine.

In some embodiments, the artificial intelligence engine may be further configured to produce the human sensory data.

In some embodiments, the artificial intelligence agent may be further configured to predict the emotional response to the external stimulus.

In some embodiments, the artificial intelligence agent may be further configured to emulate a user with a specific disposition.

In some embodiments, the artificial intelligence engine may be further configured to produce the human sensory data based on a user with a specific disposition.

In some embodiments, the artificial intelligence agent may be further configured to predict the emotional response to the external stimulus by emulating a user with a specific disposition.

In some embodiments, the external stimulus may be a form of media.

In some embodiments, the external stimulus may be an advertisement.

In some embodiments, the form of media may be at least one of a video or a film.

In some embodiments, the artificial intelligence engine may be configured to generate the human sensory data through user engagement analysis, video content analysis or natural language processing.

In some embodiments, the external stimulus may include an experience.

In some embodiments, the experience may be a process of driving a vehicle.

### BRIEF DESCRIPTION OF DRAWINGS

Reference will now be made, by way of example, to the accompanying drawings which show example embodiments, and in which:

FIG. 1 is a block diagram of a system incorporating an analytics engine, according to some example embodiments;

FIG. 2 is a block diagram of an example artificial intelligence agent;

FIG. 3 is a schematic of the analytics engine of FIG. 1, according to some embodiments;

FIGS. 4A-4B are another schematic of the analytics engine of FIG. 1, according to other embodiments;

FIG. 5 depicts an example incorporation AI agent, using the analytics system;

FIG. 6 depicts an example hierarchy of an AI system.

FIG. 7 depicts various kinds of dopamine levels;

FIG. 8 depicts an example of surprise factor segmentation;

FIG. 9 is a block diagram of an analytics system incorporating the analytics engine of FIG. 1, according to some example embodiments;

FIG. 10 is a block diagram of external stimulus in the system of FIG. 9;

FIG. 11 is a block diagram of output in the system of FIG. 9;

FIGS. 12A-12C depict examples of sense, energy levels, and action organs.

FIG. 13 is a block diagram of the analytics engine in the system of FIG. 9;

FIG. 14 is a block diagram of sensory data in the system of FIG. 9;

FIGS. 15A-15D depict examples of dopamine and serotonin concentrations;

FIGS. 16A-16C depict examples of the bandwidth for human senses;

FIG. 17 is a block diagram of acquiring the sensory data of FIG. 14;

FIG. 18 depicts an example of sensory data acquired using biometric sensors.

FIG. 19 is a block diagram of collecting, from a real audience, the sensory data of FIG. 9;

FIG. 20 depicts an example of measured sensory data, according to various external stimuli and energy level segments;

FIG. 21 is a block diagram of emulating the sensory data of FIG. 14;

FIG. 22 is a block diagram of emulating, based on an external stimulus, the sensory data of FIG. 14;

FIG. 23 is a block diagram of deriving emotional response from the sensory data of FIG. 9;

FIG. 24 is a block diagram of the sensory data of FIG. 14 comprising a plurality of time intervals;

FIG. 25 is a block diagram of predicting financial success based on the emotional response of FIG. 23;

FIGS. 26A-B depict an example of evaluating the financial success of various movies;

FIG. 27 depicts an example of a desirable emotional response;

FIG. 28 depicts an example of an undesirable emotional response;

FIG. 29 is a block diagram of predicting financial success, incorporating the analytics engine of FIG. 9;

FIG. 30 is a method for determining the change of the emotional response using the system of FIG. 14, according to some example embodiments; and

FIG. 31 is a method for determining the success using the system of FIG. 25, according to some example embodiments;

FIGS. 32A-B depict an example embodiment of a movie analytics engine, using the analytics system of FIG. 9.

FIG. 33 is a block diagram of a computing device, according to an example embodiment; and

### DETAILED DESCRIPTION

### Analytics Engine

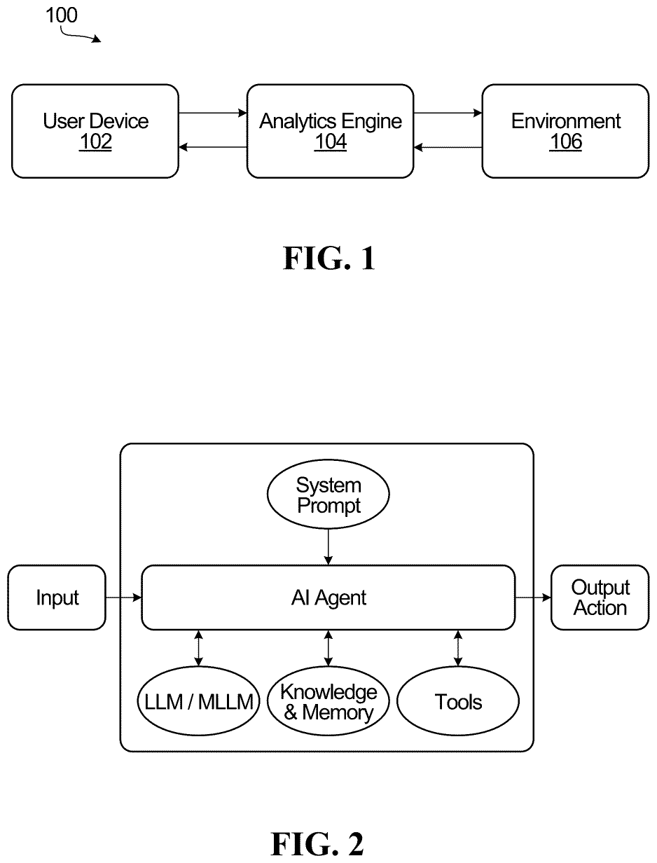

FIG. 1 depicts a system 100, which includes a user device 102, an analytics engine 104 and an environment 106.

User device 102 may a computing device, such as a mobile device, a personal computer, a server, an embedded system or some other device with computing capabilities. User device 102 may receive input from a user, such as a human, or from another computing device, such as one or more sensors, equipment and/or information systems.

User device 102 communicates with analytics engine 104, such as over a network (not depicted). User device 102 and analytics engine 104 may exchange information with one another, such that user device 102 may both transmit information to and receive information from analytics engine 104. The network may include the Internet, an intranet, a WiFi network, a Bluetooth network, an iBeacon network, or some other communication protocol which allows user device 102 and analytics engine 104 to exchange information.

In some embodiments, analytics engine 104 may be executed, hosted and/or stored on a server, multiple servers or some other computing device(s). In these embodiments, cloud computing may be used to allow user device 102 to communicate with analytics engine 104.

In some embodiments, analytics engine 104 or portions of analytics engine 104 may be executed, hosted and/or stored on user device 102. In these embodiments, edge computing or a combination of edge computing and cloud computing may be used to allow user device 102 to communicate with analytics engine 104. In the embodiments where only a portion of analytics engine 104 is executed, hosted and/or stored on user device 102, analytics engine 104 contained on user device 102 may communicate with the portion of analytics engine 104 executed, hosted and/or stored on a server or some other external computing device.

Analytics engine 104 may also communicate with environment 106, such as over a network (not depicted). Examples of a network may include the Internet, an intranet, a WiFi network, a Bluetooth network, an iBeacon network or some other communication protocol. Analytics engine 104 may query environment 106, send data to environment 106, retrieve data from environment 106 and respond to queries from environment 106. Analytics engine 104 and environment 106 may communicate bidirectionally, similar to user device 102 and analytics engine 104. As will be discussed in further detail below, environment 106 may include databases, the Internet, such as websites, application specific interfaces (APIs), computing devices, sensors, an intranet, internal systems, etc.

Analytics engine 104 may be used to process data, generate analytics insights based on data, respond to queries received from input device 102, automate tasks and processes, and/or solve problems.

In some embodiments, analytics engine 104 may provide a unified application capable of embodying Artificial Narrow Intelligence (ANI), Artificial General Intelligence (AGI), and Artificial Super Intelligence (ASI). Analytics engine 104 may be used by an organization or user of user device 102 to engineer artificial intelligence (AI) solutions and task automation.

ANI may also be known as weak AI. ANI may refer to AI that is specialized in a specific task or a narrow range of tasks. A key characteristic of ANI may a solution which is task specific, limited in scope, and without any true understanding. However, ANI may be more efficient than humans, in some examples.

AGI may be capable of understanding, learning and performing any intellectual task that a human can perform.

ASI may go further than AGI by exceeding human capabilities in every domain. ASI may create self-driven goals and/or objectives.

AGI and ASI may be capable of solving a vast array of problems across multiple domains, exhibiting versatility and adaptability. Some problems AGI and ASI may solve include complex decision-making, multitasking across domains, autonomous innovation, enhanced efficiency and productivity, improved personalization and solving global challenges.

Currently, AGI and ASI may be theoretical and elusive concepts. While ANI exists, its development may often be custom, cumbersome, and time-consuming. System 100 and analytics engine 104 may offer a streamlined solution capable of addressing and solving any problem efficiently across the spectrum of AI capabilities.

For example, existing technologies may include disparate ANI solutions such as demand prediction models, image classification models, etc. These solutions may often be limited to specific, narrow applications. Additionally, generative AI solutions like GPT-4, Gemini 1.5, and Llama 3 are available but may not be integrated into a unified system that encompasses ANI, AGI, and ASI.

In some embodiments, analytics engine 104 may also include a team of AI agents. As used herein, an AI agent may include at least one AI model, such as a large language model (LLM) or multi-model large language model (MLLM). An AI agent may also include one or more other models or tools to allow AI agent to perform one or more tasks. Furthermore, as used herein, the terms AI agent and intelligent agent may also be used to refer to a single AI agent or one or more AI agents, such as a group of AI agents which may collaborate to solve problems.

An example AI agent is depicted in FIG. 2, which may include an LLM or MLLM, knowledge and memory, and tools. The AI agent may input. In some examples, the AI agent may also include a system prompt. The AI agent may generate an output action based on the input, the LLM/MLLM, knowledge and memory, and/or system prompt.

In some examples, an AI agent may be a computational entity designed to perform tasks by perceiving inputs, processing information, and executing actions to achieve specific goals. At its core, the AI agent may include an LLM or an MLLM, which may serve as the brain of the AI agent, enabling input data and processing understanding, contextual reasoning, and decision-making. The AI agent may be equipped with tools such as task-specific APIs, plugins, or computational modules, which may extend the capabilities of the AI agent beyond language processing to include data retrieval, numerical analysis, and/or automated workflows. The AI agent may receive inputs through various connections, including natural language commands, structured data (e.g., tables or databases), sensory data (e.g., audio, video, or environmental metrics), and external APIs for real-time information. These inputs may be preprocessed in a processing layer to ensure context-aware decision-making. The AI agent may produce output actions that range from generating natural language responses to executing tasks via APIs, controlling physical devices, and/or delivering data insights and visualizations. To improve continuously, the AI agent may integrate a feedback loop and learning mechanisms, leveraging user feedback, logged interactions, and/or reinforcement learning to refine its performance over time.

As already discussed briefly above, embodiments of system 100 may include cloud, edge, and both cloud and edge computing. For example, in some embodiments of cloud computing, edge computing, and both cloud and edge computing, predicative AI, generative AI and an agentic framework with a team of AI agents may be used. These embodiments may be used to achieve ANI/AGI/ASI.

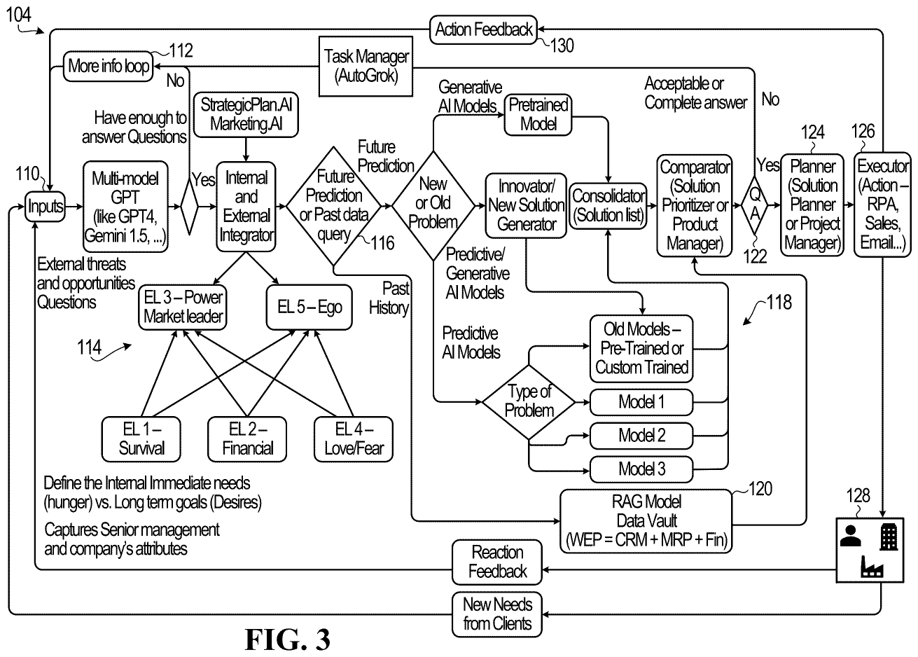

FIG. 3 depicts a schematic of analytics engine 104, according to some embodiments.

Analytics engine 104 includes inputs 110, which may be received from user device 102 and/or environment 106. Inputs 110 may include information, data, queries, prompts, problems to be solved and/or other input. Inputs 110 may be in the form of text, images, videos, a stream, documents and/or some other data format.

In some embodiments, inputs 110, which may include data and/or one or more prompts, may be passed on to every step of analytics engine 104 discussed below. Passing inputs 110 to every step may allow every step in analytics engine 104 to decide if any portion of inputs 110 is relevant to that step, and this may reduce time for analytics engine 104 to respond to inputs 110. Steps in analytics engine 104 may include AI agents and/or LLMs in analytics engine 104, which are discussed in further detail below.

Analytics engine 104 may also include more information loop 112. At more information loop 112, analytics engine 104 may determine whether inputs 110 is sufficient for analytics engine 104 to provide a result, solution or answer, such as an answer to a query or a solution to a problem. If inputs 110 is not sufficient, such as if more information or data is required, more information loop 112 may request more information, data and/or other input at inputs 110.

Analytics engine 104 may also include AI agents 114. In some embodiments, AI agents 114 may define the internal immediate needs (hunger) versus long term goals (desires) of an organization, business or some other entity. For example, AI agents 114 may capture the attributes of senior management in an organization/company and the company itself.

Analytics engine 104 may include a decision point 116, which may determine whether inputs 110 relate to future prediction or a query related to past history. Decision point 116 may connect to a future prediction module 118 if inputs 110 relate to future prediction. Decision point 116 may instead connect to a past history query module 120 if inputs 110 relate to a query of past history. In some examples, decision point 116 may connect to both future prediction module 118 and past history query module 120, such as in examples where inputs 110 relate to both future prediction and a query related to past history.

Future prediction module 118 may assess whether a problem specified by inputs 110 is a new problem or an old problem. Depending on whether the problem is new or old, a pretrained model may be used, which may be a generative AI model or models. As well, if the problem is old, the type of problem may be identified and one or more old, pre-trained or custom trained models may be used. If the problem is new, predictive and/or generative AI models may be used to generate or select one or more pre-trained or old models. In some examples, multiple problems may be included in inputs 110 and/or a single problem may require multiple models, and so the outputs of multiple models may be consolidated into a solution list at future prediction module 118.

Past history query module 120 may include a retrieval-augmented generation (RAG) model and/or data vault. Past history query module 120 may also include resources for enterprise resource planning (ERP), including resources customer relationship management (CRM), material requirements planning and financials.

Analytics engine 104 may also include comparator 122. At comparator 122, an output solution or solutions from one or both of future prediction module 118 and past history query module 120 may be assessed to determine if the solution or solutions provide an acceptable or complete answer to the problem or queries included within inputs 110. If the solution or solutions are not acceptable or complete, comparator 122 may loop back to an earlier stage within analytics engine 104 to repeat or refine the solution generation process, such as by requiring more data or information at inputs 110. If the solution or solutions are acceptable or complete, comparator 122 may proceed.

As a precursor to execution, analytics engine 104 may also include planner 124, which may include a solution planner or project manager. Planner 124 may break down the solution into smaller steps, if needed, before execution. Planner 124 may include the solution or solutions from comparator 122.

Analytics engine 104 may also include executor 126, which may perform actions based on planner 124. Actions may include a computer action like sending emails, performing or coordinating sales, robotic process automation (RPA), etc.

Analytics engine 104 may communicate with environment 106, as already discussed above. In some examples, environment 106 may include company infrastructure, systems, computing devices, vendors, websites, etc. Executor 126 may perform actions to environment 106. Analytics engine 104 may also receive feedback from external feedback mechanism 128, which may be connected to company or organization infrastructure, such as within environment 106. Feedback may include reaction feedback, new needs from clients (e.g. clients of the company or organization or the organization itself) or other forms of feedback. This feedback may be fed back into inputs 110, which may be used in another process loop of analytics engine 104 or considered for further processing.

In addition, action feedback 130 may be generated by executor 126 for analytics engine 104 to feed into inputs 110 on a subsequent process loop or consider for further processing.

It will be understood that other embodiments and examples of analytics engine 104 may also be possible. Some or all of the modules or stages discussed above within analytics engine 104 may be rearranged, removed or replaced, and new or other modules not discussed so far may also be included within analytics engine 104.

Analytics engine 104 may integrate predictive AI, generative AI, and agentic AI workflows. As will be discussed further below, analytics engine 104 may employ a team of AI agents alongside a phone application for data input (e.g. user device 102). This computation may then be executed in the cloud, on edge devices, and/or a combination of both.

Data Integration and Sources: Analytics engine 104 may connect AI agents to various data sources, such as ERP, CRM and financial systems (e.g. within environment 106). Analytics engine 104 may facilitate seamless data flow and AI configuration. Data may also be collected from noise, vibration, harshness (NVH), global positioning system (GPS), voice, and vision sensors embedded in a phone, such as a phone providing input to analytics engine 104 (e.g. user device 102). This data may be used for training custom models or for real-time inferencing to predict future outcomes.

Holistic Application Functionality: Analytics engine 104 may enable comprehensive analysis by examining historical data to answer questions about past events. Predictions may be generated using pre-trained and/or custom-trained models, which may be deployed either in the cloud or on edge devices.

Feedback Loop and Continuous Improvement: A feedback loop may be integrated in analytics engine 104 for handling unsolved or partially solved problems. Even fully resolved issues may remain open until the corresponding reactions or outcomes are recorded, which may ensure continuous improvement and accuracy.

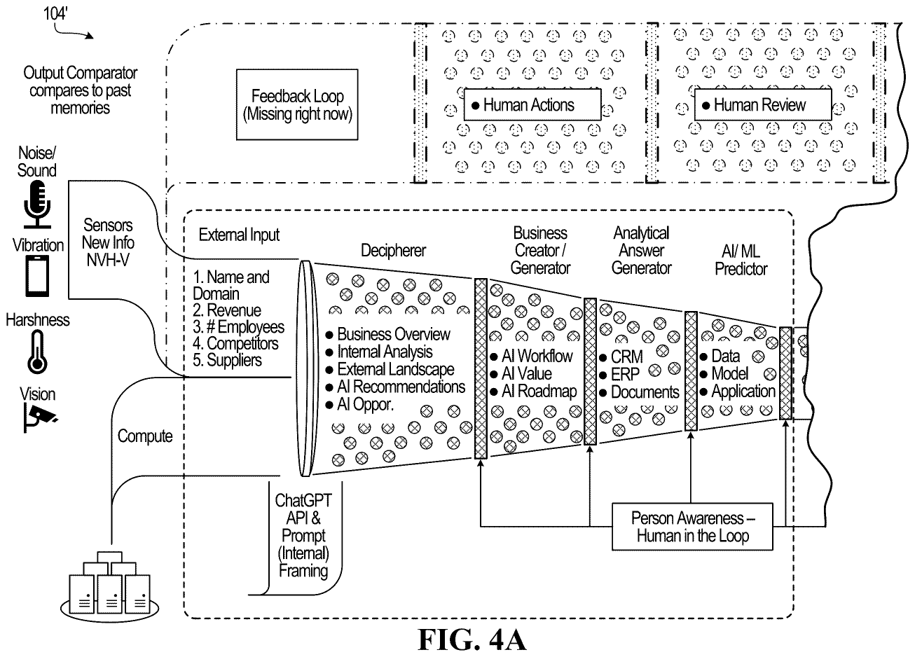

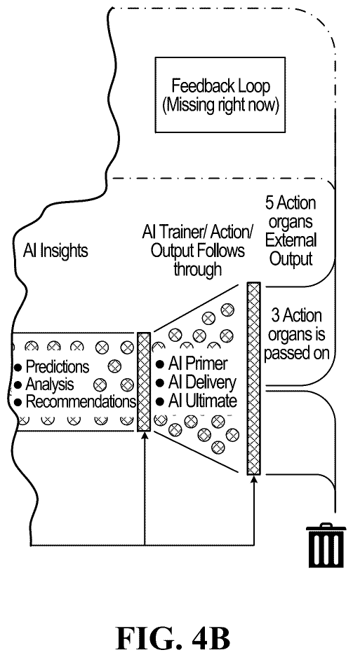

FIGS. 4A-4B depict an analytics engine 104′, according to some other embodiments of analytics engine 104. It will be understood that analytics engine 104 and analytics engine 104′ may be interchangeable in system 100, and all reference to analytics engine 104 as used herein may also refer to analytics engine 104′.

Analytics engine 104′ may receive external input. In some examples, external input may include sensor information and new information. Sensor information may include noise/sound, vibration, harshness and vision (NVH-V) information.

In further examples, external input may also or instead include information describing a company, such as name and domain information, revenue of the company, a number of employees at the company, competitors of the company, customers of the company and suppliers of the company.

External input may also or instead include computation data and/or prompt data. Prompt data may be parsed by a large language model, such as by an API, e.g. the ChatGPT™ API.

Analytics engine 104′ may include a decipherer external input. Decipherer may generate a business overview, internal analysis, external landscape, AI recommendations and/or AI opportunities identified by analytics engine 104′. Decipherer may also store its inputs and outputs in a memory of analytics engine 104′.

It will be appreciated that memory and awareness may be important determinants in decision making. Memory may be akin to weights and biases in a pre-trained AI model. Awareness may be akin to a processor. Memory and awareness may be found in reinforcement learning from human feedback (RLHF), which may be human awareness laced and may benefit from a good pre-trained model.

Analytics engine 104′ may also include a business creator/generator, which may generate an AI workflow, AI value and/or AI roadmap. Business creator/generator may also store its inputs and outputs in a memory of analytics engine 104′.

Analytics engine 104′ may also include an analytical answer generator, which may receive and/or generate CRM, ERP and documents. Analytical answer generator may also store its inputs and outputs in a memory of analytics engine 104′.

Analytics engine 104′ may also include an AI/machine learning (ML) predictor, which may receive and/or generate data, models and software applications. AI/ML predictor may also store its inputs and outputs in a memory of analytics engine 104′. As used herein, the term artificial intelligence (AI) also includes machine learning.

Analytics engine 104′ may also generate AI insights, which may include predictions, analysis and/or recommendations. AI insights may also be stored in a memory of analytics engine 104′.

Analytics engine 104′ may also include an AI trainer and/or may perform actions on output. As used herein, an artificial intelligence engine may receive and/or generate data, models and/or other content.

Action organs and external output may be passed on in a feedback loop, as well as with human actions to the output. The feedback loop may include a comparator, which compares the output to past memories, e.g. the memories of analytics engine 104′. The feedback loop may return to the input and also be fed into analytics engine 104′ as external input. In other examples, the output may be discarded by analytics engine 104′.

It will be understood that other embodiments and examples of analytics engine 104′ may also be possible. Some or all of the modules or stages discussed above within analytics engine 104′ may be rearranged, removed or replaced, and new or other modules not discussed so far may also be included within analytics engine 104′.

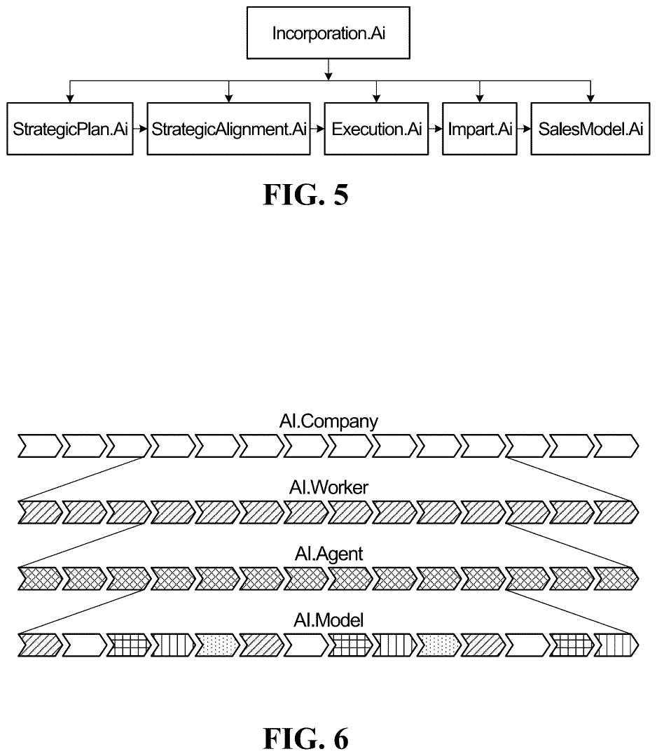

In addition, system 100 may include or interface with one or more modules and/or AI agents, for instance as part of another system. For example, as depicted in FIG. 5, system 100 may be used by or communicate with an incorporation AI agent, which may communicate with other AI agents. For instance, the incorporation AI agent may communicate with a strategic plan AI agent, a strategic alignment AI agent, an execution AI agent, an impart AI agent, and/or a sales model AI agent.

Moreover, as depicted in FIG. 6, system 100 may be used by or communicate with an AI company. The AI company may include one or more AI workers, which may each include one or more AI agents. Each AI agent may include one or more respective AI models and/or tools (e.g. retrieval-augmented generation agent, web crawler, etc.). The AI models may be pre-trained (e.g. an LLM, DocAI™, Route-AI™, etc.), custom trained (e.g. image classification), and/or deployed in the cloud for use with an API handshake. The output of AI workers may be fine-tuned based on an audience.

Other examples of an analytics engine are also described in U.S. patent application Ser. No. 19/028,962 filed on Jan. 17, 2025, entitled “System and Method for Planning with Artificial Intelligence”, which claims the benefit of U.S. Provisional Patent Application No. 63/667,639 filed Jul. 3, 2024, U.S. patent application Ser. No. 19/029,167 filed on Jan. 17, 2025, entitled “System and Method for Monitoring with Artificial Intelligence”, which claims the benefit of U.S. Provisional Patent Application No. 63/667,639 filed Jul. 3, 2024, are also all incorporated herein by reference in their entirety.

### Technological Context and Considerations

In existing solutions, product or service providers may attempt to quantify the success of a user's interaction with a product or service by requesting that the user complete a survey and/or submit a review with a rating (e.g. 4/5 stars). In response to such feedback, a product or service provider may adjust the product or service and monitor subsequent outcomes. Providers may also merely use sales data as a proxy for the success of the user's interaction. However, these approaches may present several limitations.

For example, providers may be unable to directly measure the user's emotional response to the product or service, may lack tailored recommendations for improvement, and may only receive feedback after the interaction has occurred (e.g. ratings or sales data). As a result, providers may not be able to predict the user's response and/or feedback about the product or service, which may be undesirable.

Furthermore, reliance on a test user base may constrain the ability to capture responses from intended target demographics. The number of variations that can be trialed may also be limited, and the data collected may be insufficient to support meaningful conclusions.

Embodiments disclosed herein may address one or more of the above limitations by enabling the measurement or quantification of human interactions, such as with products, services, and experiences, based on emotional responses. Embodiments disclosed herein may enable the processing of multi-modal sensory data in a time-variant manner to compute the emotional responses and may improve human-computer interaction and/or emotion modelling.

In some embodiments, a real-time feedback loop may be used to adapt the product, service, or experience based on the interaction and the measured emotional response, providing a technical improvement over existing methods described above. Embodiments disclosed herein may also provide methods to predict an emotional response for different demographics by emulating users with a specific disposition.

### Interactions and Emotional Response

Interactions may be enigmatic and present information exchange may be a “black box”. Knowing what information is sent may be easier than determining what is perceived or received. Determining the emotional response to a stimulus (e.g. a product, event, experience and/or service) may depend on several factors. For instance, an individual's interaction with a product, event, experience and/or service may vary depending on the individual's specific individual characteristics and dependent upon the individual's expectations, as well as on the form of the product or service with which the individual is interacting. Interactions and emotional responses may differ among entities, e.g. an AI to a human, a human to an AI, an AI to another AI. Other types of entity interactions may also be possible.

As well, as used herein, the terms product, event, experience, and service may describe media (e.g. videos, pictures, social media posts), vehicles, travel experiences, locations, social interactions (e.g. restaurant service, a customer service call, etc.) and any other stimulus which cause an emotional response in an individual.

In some embodiments, determining the context may be necessary to understand an interaction with a product, service, or experience. Context may be set by residual chemicals, such as dopamine and cortisol, which may have a gradual decline over time. For example, if someone demeans another person, that person's day may be ruined and may provide context for understanding the success of a product, service, or experience.

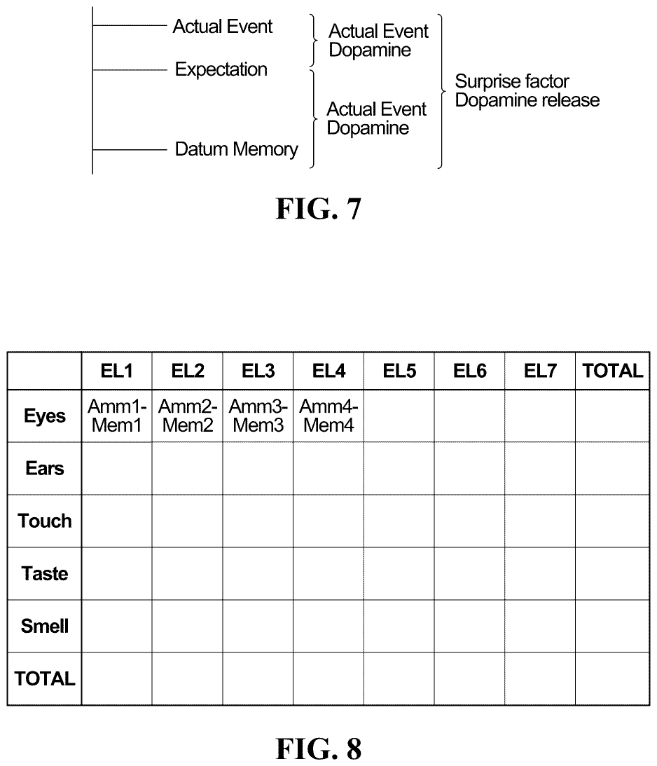

In some embodiments, dopamine levels may be measured for a user experiencing or interacting with a product or service. A user may experience a dopamine peak, crash, deficit, and/or may return to their baseline dopamine level over time in response to a stimulus. For instance, a comedy experience or event (e.g. a comedy film/movie or act) may cause a surprise that leads to a high level of dopamine secretion. Similarly, the anticipation of a positive event may also lead to dopamine secretion, such as listening to music or shopping for some users. Anticipation dopamine may be based on the expectation of a user, as depicted in FIG. 7. Surprise factor (referred to herein as an “S” factor) dopamine may be based on the anticipation dopamine and the actual event dopamine.

In some cases, such as in a movie or a book, having closure or a resolution to the story may satisfy a user and/or relax the user's mind. For example, good stories may follow Freytag's Pyramid, where a story begins at a “low” point, rises towards a climax as tension, conflict, and challenges are presented. After the climax, a story may “descend” as conflicts resolve and may ultimately reach a conclusion, leaving the audience at a new equilibrium or a point of reflection. Successful movies may also include a main character with a charismatic personality that captivates viewers.

Successful products and services may share certain qualities with successful movies. For example, they may impact various parts of the brain, they may create expectations and may meet or exceed those expectations, and they may start from a low point and rise to a higher point.

Moreover, successful movies may result in an emotional response of a user that is characterized by one or more sigmoidal curves during the time period of the user watching the movie. An emotional response sigmoidal curve may be at a low value at one point and rise to a higher value at a later time during the interaction with the external stimulus (e.g. watching the movie). Sigmoidal curves may be successive in nature and may represent a desired emotional response. For example, having a number of surprises throughout a movie may result in a more successful response.

In some embodiments, as disclosed herein, the dopamine level of a user may be used as a proxy for the emotional response. For example, the emotional response may be based on the “S” factor dopamine release of a user. A high “S” factor dopamine level of a user with a sigmoidal curve during the period of time may result in a higher emotional response.

The “S” factor of a user may also be based on other sensory measurements, in addition to dopamine secretion. For example, serotonin levels of a user may be measured and/or estimated and used to determine the “S” factor. In some cases, the serotonin level may be predicted or estimated using an AI agent. Other senses, including sight, hearing, touch, taste, and smell may be measured and used for computing the “S” factor. Other measurements may also be used. There may be variation among users with respect to sensory measurements and emotional response to various products or services.

In some embodiments, the “S” factor may be personalized to a user. For example, for a user with a stronger incline to human romance, there may be a higher “S” factor versus a user who may have a weaker incline to human romance, such as in the context of determining an emotional response to romance movies.

The emotional response, using the “S” factor as a proxy, may be assigned a numeric value. In some embodiments, the emotional response may be quantified using vector embeddings or numbers.

The “S” factor for a user may be determined for a particular energy level. For example, energy levels may include security (EL1), pleasure (EL2), focus and competence (EL3), courage and lack of fear (EL4), communication and ego (EL5), and compass, e.g. vision driven by purpose and global perspective (EL6); however, other variations of energy levels may be possible and may have different “EL” labels assigned. Determining the “S” factor with respect to a particular energy level may provide the necessary context for determining the emotional response and/or the success of the product or service. For instance, a product provider may only care about the success of a product for a certain energy level. In some embodiments, the energy levels may also be referred to as emotional landscapes. Energy levels may also be mapped to the Maslow Hierarchy of needs.

In some embodiments, the “S” factor over time for a user's response to a product, event, experience or service may be used to determine the success for that product, event, experience or service. FIG. 8 depicts an example for calculating “S” factors through segmentation for different senses and energy levels. In some embodiments as disclosed herein, the derivative of the “S” factor with respect to time may be computed and used to determine the success of a product, event, experience or service, including the financial success, engagement level, or loyalty change.

In determining the success of a product, event, experience or service, a user's emotional response or change of emotional response may be compared to a desired emotional response or desired change of emotional response, respectively. The desired emotional response may be a sigmoidal curve which is at a low value at one point and rises to a higher value at a later time during the interaction with the external stimulus. However, it is possible that the desired emotional response may have other curve characteristics.

The desired emotional response could be specific to an energy level, user profile, or other factor. For example, the desired emotional response used for comparison may be different for a comedy versus an action movie. Similarly, the desired emotional response may depend on user demographics and other objectives. The desired emotional response may also include or describe a power, an ego and/or a global compass response.

### Example Analytics Engine for Emotional Response

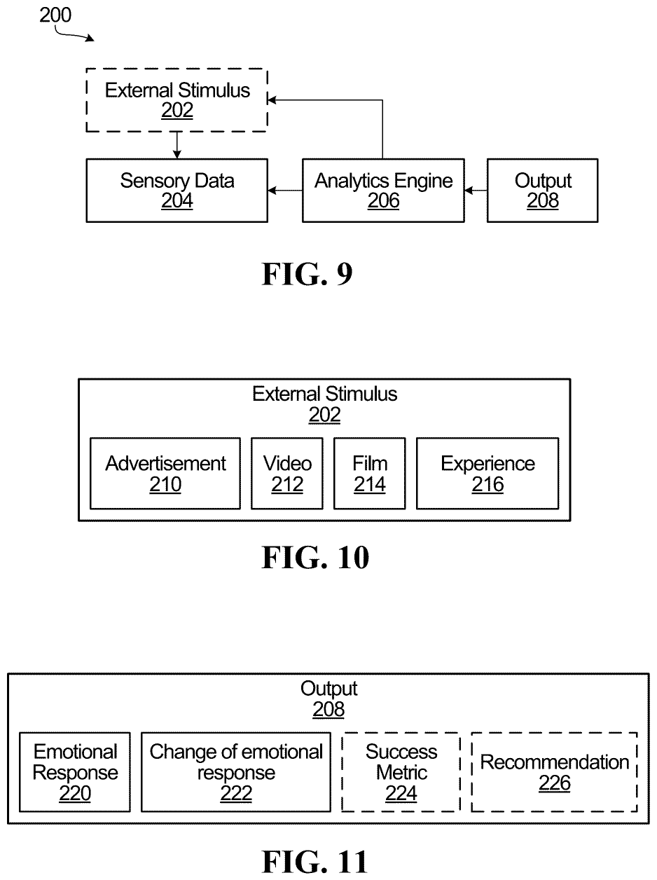

FIG. 9 depicts a system 200, which may be used to determine an emotional response. System 200 includes sensory data 204, an analytics engine 206, and an output 208. Optionally, system 200 may include an external stimulus 202. System 200 may be used by a company or business. Analytics engine 206 may be the same as or similar to analytics engine 104 and/or analytics engine 104′ described above.

FIG. 10 depicts the external stimulus 202, which may be or include a form of media, including an advertisement 210, video 212, and/or a film 214. External stimulus 202 may also into text, an image, audio, and/or other media. In some embodiments, external stimulus 202 may be adapted based on analytics engine 206. For instance, the analytics engine may modify, revise, delete from, add to, or re-generate external stimulus 202. External stimulus 202 may also or instead include other forms of media.

In some examples, external stimulus 202 may be or include an experience 216, such as a process of driving a vehicle, riding a roller coaster or some other stimulus causing sensory data to be generated. External stimulus 202 may also or instead include other types of stimuli.

As depicted in FIG. 11, output 208 of analytics engine 206 may be an emotional response 220 and/or a change of emotional response 222. Emotional response 220 may be determined by analytics engine 206 based on sensory data 204. A numerical value may be assigned to emotional response 220. For instance, the “S” factor over time for a user's response to external stimulus 202, based on sensory data 204, may represent or be used as a proxy for emotional response 220.

In some embodiments, output 208 may also include the derivative of the change of emotional response and/or the predicted financial success of the media (e.g. advertisement 210, video 212 or film 214, and/or text, image, audio, etc.) or experience 216. In some embodiments, output 208 may provide feedback to analytics engine 206 to determine the derivative of change of emotional response. For example, the derivative of the “S” factor with respect to time may be computed to represent the derivative of change of emotional response 222.

In some embodiments, output 208 may provide feedback to analytics engine 206 to determine a second derivative of the change of emotional response 222.

In another embodiment, change of emotional response 222 may refer to a difference with respect to a defined threshold.

Determining emotional response 220 may also include a mapping or classification of external stimulus 202 to an energy level. Classification of external stimulus 202 may provide context for determining emotional response 220 and/or the success of external stimulus 202 in providing a desirable emotional response 220. Classification may be performed by analytics engine 206 using one or more AI agents. As noted above, an AI agent may include one or more LLMs and/or other software modules, tools, models, etc. In some further embodiments, classification may also be performed using one or more disposition profiles, each disposition profile representing an example person with certain characteristics or a certain disposition (e.g. a young man, an elderly woman, a doctor, a child), such that the success of external stimulus 202 is assessed relative to the perspective of that example person. Classification with disposition profiles may be performed using one or more AI agents. In some other embodiments, mapping may also be performed manually.

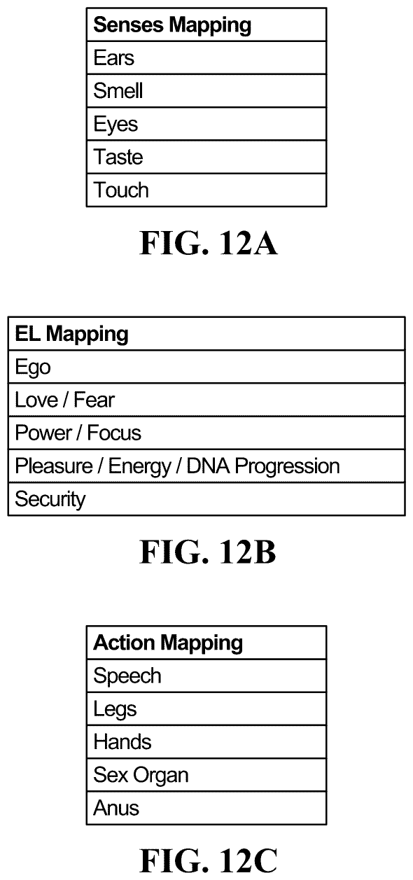

FIGS. 12A-C depict examples of various input senses, energy levels, and action organs. For example, as depicted in FIG. 12A, ears, smell, eyes, taste, and/or touch may provide input senses that may generate measured sensory data 204 and/or a corresponding sentiment curve in response to a trigger. Moreover, FIG. 12B depicts five possible energy levels, e.g. ego, love/fear, power/focus, pleasure/energy/DNA progression, and/or security, which may be invisible drivers that act as a black box. Each of these energy levels may be housed within the human brain and may provide a corresponding sentiment curve in response to a trigger, e.g. an external stimulus.

FIG. 12C depicts various output action organs, e.g. speech, legs, hands, sex organ, and/or anus, which may provide corresponding sentiment curves. For instance, output organs may only trigger when a human acts in response to a stimulus, whether the stimulus is an external stimulus or from an internal bodily function. In some cases where a human is a passive observer, the action organs may not be triggered. In other cases, the action organs may trigger when there is a reaction to a threat, opportunity, and/or a fight or flight reaction. A human's focus and/or awareness may be tied into and/or related to one or more input senses, energy levels, and/or action organs at a given moment. In some embodiments, the sex organ, anus, and/or other action organs may not be relevant externally or provide a useful sentiment curve in response to a trigger.

In some embodiments, output 208 may also include a success metric 224. Success metric 224 may be a prediction of financial success, such as the predicted revenue, gross profit, or net income. For example, external stimulus 202 may be a form of media and success metric 224 may be determined by analytics engine 206, and analytics engine 206 may describe the predicted success of the media (e.g. financial success). In other embodiments, success metric 224 may describe other aspects of external stimulus 202, such as a ranking or rating, including with respect to other external stimuli.

Success metric 224 may also be an engagement level or loyalty change. In another example, external stimulus 202 may be an experience 216, e.g. driving a new car model, flying a plane or dining at a certain restaurant. Success metric 224 may be determined by analytics engine 206. In the example experience 216 of driving a new car model, success metric 224 may represent the predicted increase in loyalty of a user after buying the new car. In this example, external stimulus 202 may be a specific experience associated with the new car model, such as sitting in the driver's seat, opening the trunk, or backing up out of a driveway.

Output 208 may further include a recommendation 226. Analytics engine 206 may generate recommendation 226 based on success metric 224 associated with external stimulus 202, which may be used to adapt external stimulus 202. For example, recommendation 226 may provide instructions to analytics engine 206 to adapt one or more aspects of external stimulus 202 to generate new sensory data 204 that will result in a desired success metric 224. Analytics engine 206 may output a new success metric based on the adapted external stimulus 202 and generated sensory data 204.

Alternatively, recommendation 226 may provide instructions to analytics engine 206 to adapt one or more aspects of sensory data 204 that will result in a desired success metric. For example, adapting sensor data 204 may include generating an external stimulus that produces said sensory data. Analytics engine 206 may continue analyzing external stimulus 202 and/or sensory data 204 any number of times and/or frequency, which may depend on recommendation 226 and/or success metric 224.

In some further embodiments, output 208 may also include new content generated by analytics engine 206, such as based on a sigmoidal curve. The new content may include text, image, video and/or other media. The desired emotional response of the new content may begin at a low value at the beginning of a period of time and may rise to a higher value at the conclusion of the period of time, as described above. This desired emotional response may be used to create the new content to have a higher stickiness factor for audiences.

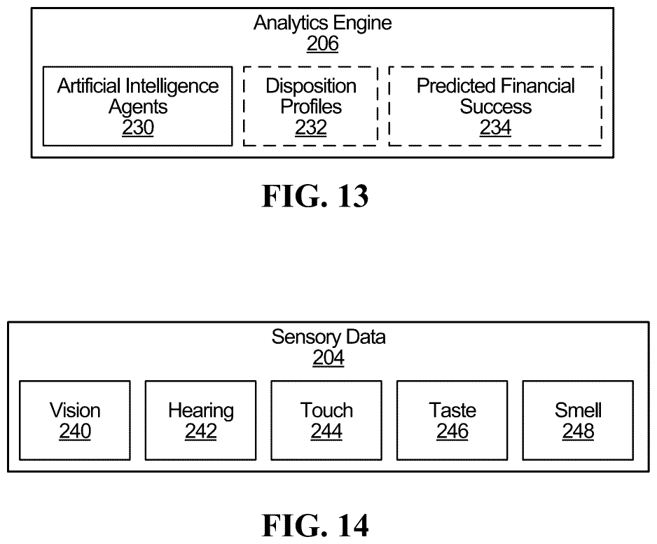

FIG. 13 depicts analytics engine 206, according to some embodiments. In some embodiments, analytics engine 206 may be the same or substantially similar to analytics engine 104 and/or analytics engine 104′.

Analytics engine 204 may include one or more AI agents (individually and collectively referred to herein as AI agents 230). For example, analytics engine 206 may only include a single AI agent 230 to compute the change of emotional response 222 in response to receiving external stimulus 202. In other examples, analytics engine 206 may include a plurality of AI agents 230, and the plurality of AI agents 230 may operate together to perform analytics for analytics engine 206. AI agents 230 may be the same or similar to AI agents 114 depicted in FIG. 2 and/or FIG. 3.

AI agents 230 may be trained to emulate a user with a specific disposition. For example, analytics engine 206 may optionally include disposition profiles 232. Disposition profiles 232 may include one or more data sets for one or more AI agents 230 to be trained upon to emulate one or more users with a specific disposition.

Disposition profiles 232 may include data sets defining at least one of age, gender, knowledge, lifestyle, and/or preference. Disposition profiles 232 may also include users with specific emotional responses and/or sensory data. In some other embodiments, disposition profiles 232 may include AI models already trained to emulate a user with a specific disposition (e.g. a user with a specific age, gender, knowledge, lifestyle, and/or preference).

Analytics engine 206 may compute emotional response 220 and/or the “S” factor for a user, based on the specific disposition. For example, for a disposition profile of user(s) with a weaker response to human love, analytics engine 206 may compute a lower “S” factor for a romance movie scene.

AI agents 230 may communicate with these models in disposition profiles to emulate a user with a specific disposition, such as generating sensory data 204 based on external stimulus 202. Other examples are also possible, such as disposition profiles already being integrated in AI agents (e.g. AI agents are already trained to emulate one or more users with a specific disposition).

Optionally, analytics engine 206 may generate a predicted financial success 234. Analytics engine 206 may generate predicted financial success 234 associated with external stimulus 202 based on at least one of emotional response 220 or change of the emotional response 222. Analytics engine 206 may use AI agents 230, trained to emulate a user with a specific disposition, to generate predicted financial success 234.

It will be understood that analytics engine 206 may include fewer or more modules or components than analytics engine 104 and/or analytics engine 104′. Analytics engine 206 may also or instead include other modules or components in addition to or instead of existing modules or components in analytics engine 104 and/or analytics engine 104′.

As used herein, an AI agent may refer to a single AI agent, which may include an LLM or multi-model large language model (MLLM), with a model or tool used to perform one or more tasks for analytics engine 206. An AI agent may also refer to a group of AI agents which may be used to perform one or more tasks for analytics engine 206. Thus, the terms AI agent and group of AI agents may be used interchangeably herein.

Moreover, an AI agent may include at least one AI model, such as an LLM or MLLM. An AI agent may also include one or more other models or tools to allow AI agent to perform one or more tasks, such as for analytics engine 206. It will be appreciated that the terms LLM and MLLM as used herein may be used interchangeably.

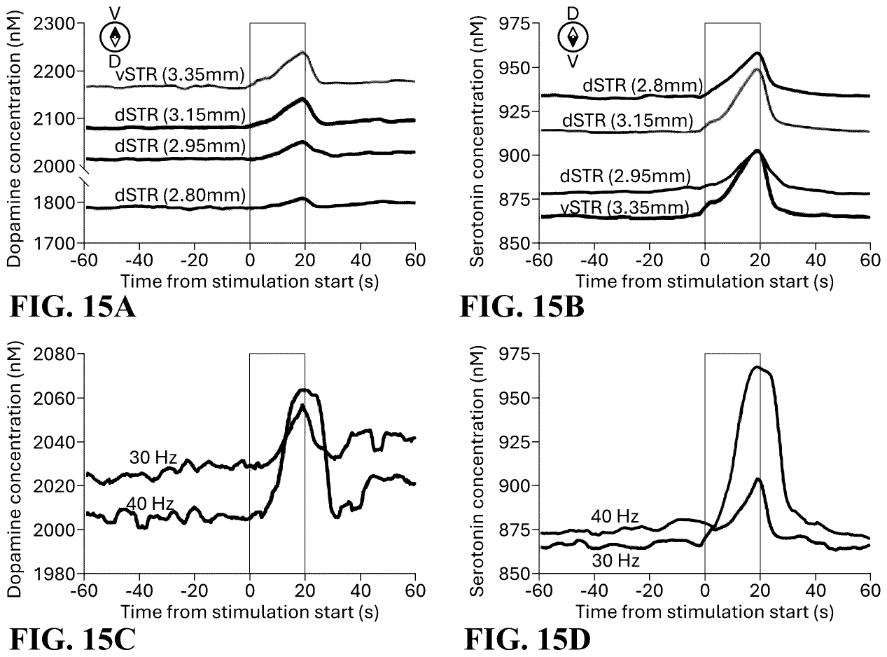

As depicted in FIG. 14, sensory data 204 may include human sensory data, such as data describing vision 240, hearing 242, touch 244, taste 246, and smell 248. Data describing other sensory data may also be included in sensory data 204. Sensory data 204 may also include, describe or be used to determine a dopamine concentration and/or serotonin concentration, including as a function of time, as depicted in the examples of FIGS. 15A-15D. Sensor data 204 may include human sensory data, and the terms may be used interchangeably herein.

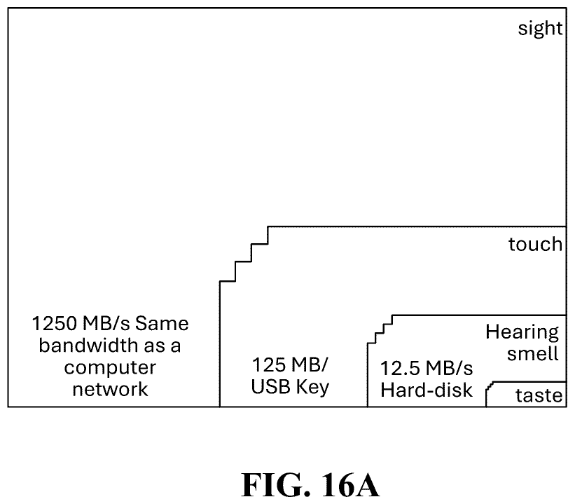

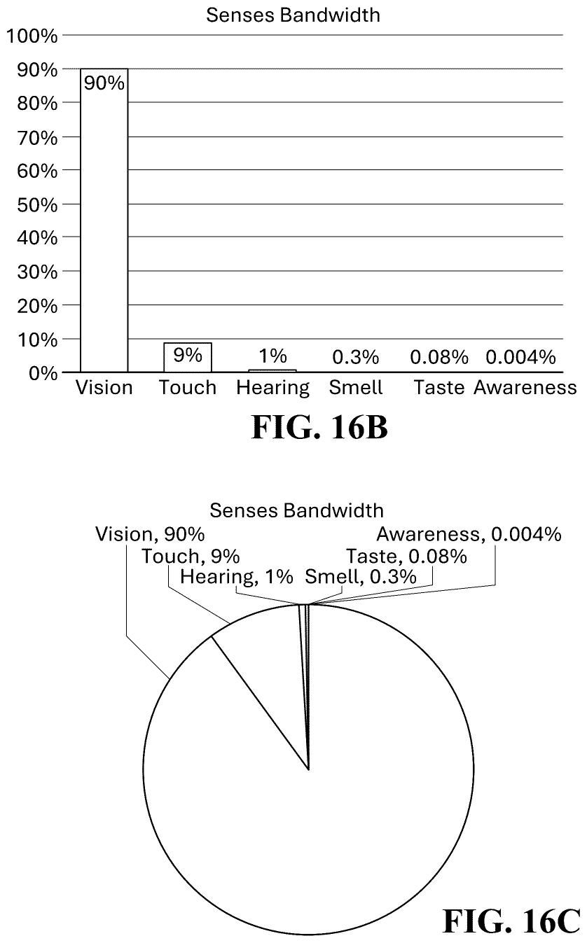

Sensory data 204 may be measured, produced by an AI (e.g. AI agent), and/or manually entered by a person. Different types of sensory data 204 (e.g. human sensory data) may have an associated bandwidth, such as depicted in FIGS. 16A-16C. For example, the bandwidth may describe the required hardware data to measure and/or stream sensory data 204 (or some aspects of sensory data 204, e.g. vision 240) or the relative data quantity sensory data 204 (e.g. vision 240 consumes or makes up 90% of measured data, while touch 224 only consumes or makes up 9% of measured data). Analytics engine 206 may use the bandwidth of sensory data 204 when computing emotional response 220.

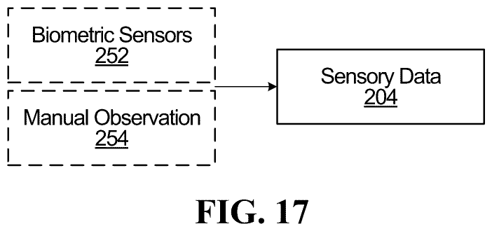

In one embodiment, sensory data 204 may be collected by biometric sensors 252 or by manual observation 254, as depicted in FIG. 17. Biometric sensors 252 may include measuring a heartbeat, the Galvanic skin response, inflammation using fingerprint expansion, piloerection (e.g. goosebumps), and/or eye-ball dilation.

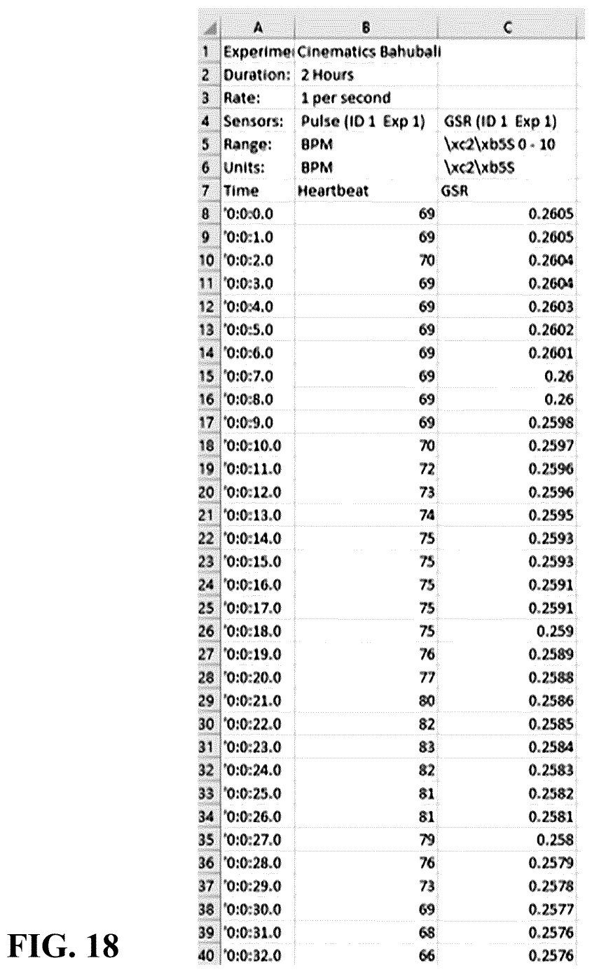

For example, a Galvanic skin sensor may be applied to one or more fingers or other areas of the skin to measure the skin surface conductivity, which may change due to the secretion of sweat glands and may change in response to external stimulus 202. FIG. 18 depicts an example of the measured Galvanic skin response and heartbeat in response to watching a movie.

As another example, goosebumps may be measured, which may change in response to an electrical signal from the hippocampus to the skin (e.g. nerve ending on the hand and sometimes legs). Goosebumps may extend to piloerection as a reaction to hearing nails scratch on a chalkboard, listening to inspiring music, or feeling and/or remembering strong or positive emotions.

Other biometric sensors 252 and methods of measurement may be used to acquire human sensory data 204. For example, modules, multi-sensors, and logger sensors may be used in any combination for acquiring human sensory data 204. Modules and/or multi-sensors may include WiFi Communication Modules, Panda Multi-Sensor, Graphic Display Module, USB Module, Battery Module, RF Communication Module, and/or a Digital Display Module. Logger sensors may include voltage, current, temperature, light, oxygen, pH, relative humidity, heart rate & pulse, photo gate, pressure, force, sound, motion, magnetic field, conductivity, spirometer, electrocardiogram, colorimeter, CO₂, Barometer, Blood Pressure, drop counter, flow, force plate, rotary motion, acceleration, salinity, soil moisture, UVB, turbidity, UVA, surface temperature, wide range temperature, infrared thermometer, respiration monitor Belt, hand dynamometer, calcium, chloride, ammonium, nitrate, anemometer, GPS, dew point, charge, Geiger counter, mA current, and/or resistance logger sensors.

In some embodiments, a mapping function, algorithm, or AI model (e.g. one or more AI agents 230) may be used to translate data acquired from biometric sensors 252 to obtain sensory data 204.

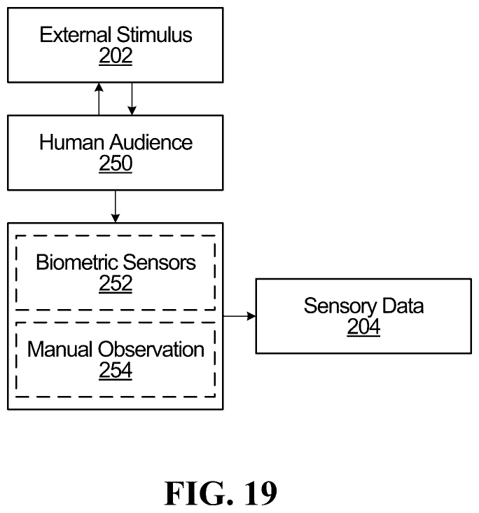

Sensory data 204 may also be acquired from a human audience 250, such as using biometric sensors 252 and/or manual observation 254, as depicted in FIG. 19. Sensory data 204 obtained from human audience 250 may change based on external stimulus 202 and may be used as training and/or input data for AI agents 230.

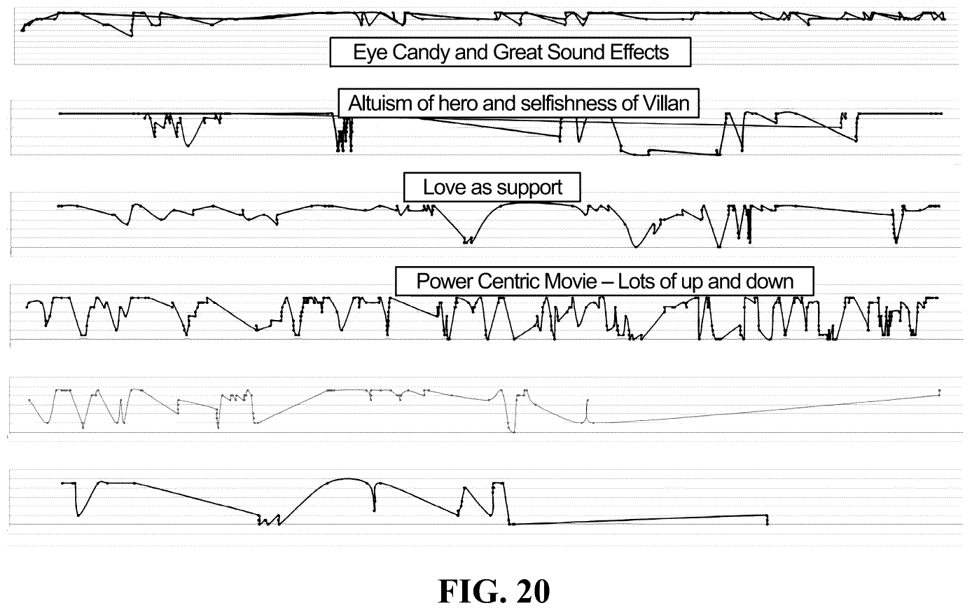

FIG. 20 depicts an example of measured sensory data 204 of a human, according to various external stimuli 202, and in particular depicts different plot characteristics with respective energy levels measured throughout a movie.

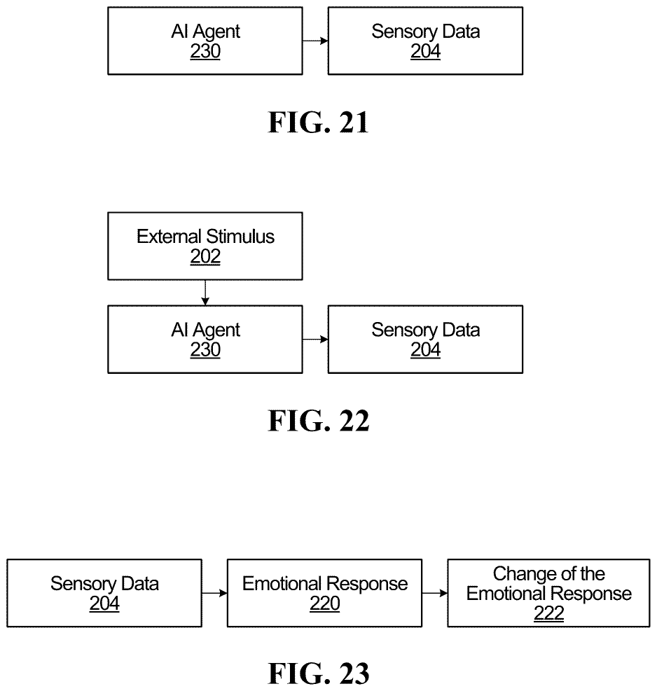

In another embodiment, sensory data 204 may be produced by AI agents 230, as depicted in FIG. 21. In some embodiments, the AI agents 230 may be trained to emulate a user with a specific disposition. As used herein, the term “produced” may also mean simulated, generated, artificially generated and/or fabricated. Sensory data 204 (e.g. human sensory data) may also be collected from AI agents 230.

Analytics engine 206 may cause external stimulus 202 to be generated, which may be used to generate sensory data 204. For example, external stimulus 202 may be generated randomly, based on a saved template, based on some other input values, and/or using any number of other methods.

In yet another embodiment, AI agents 230 may interpret external stimulus 202 and emulate how a specific user with a specific disposition may respond, as depicted in FIG. 22. For example, AI agents 230 may generate sensory data 204 for a user with a specific disposition, such as using disposition profiles 232. In a particular example, AI agents 230 may generate sensory data 204 in response to driving a new car model. Analytics engine 206 may use the emulated sensory data 204 to predict success metric 224 (e.g. satisfaction, loyalty change etc.), generate predicted financial success 234 and/or generate recommendation 226.

In another example, sensory data 204 may be produced by analytics engine 206 through user engagement analysis, video content analysis natural language processing, image content analysis, audio content analysis or video content analysis. The emulated sensory data 204 may be provided as feedback to analytics engine 206.

Analytics engine 206 may optionally use recommendation 226 and/or success metric 224 to adapt external stimulus 202 (e.g. change the appearance of the new car model) and/or recommend an adaptation to external stimulus 202. For instance, analytics engine 206 may modify, revise, delete from, add to, or re-generate external stimulus 202 (e.g. recommend a change to the colour of the new car model or modify the length of a media in external stimulus 202). Adaption, re-generation and/or recommendation of adaptation of external stimulus 202 may be performed by analytics engine 206.

As depicted in FIG. 23, sensory data 204, including simulated/emulated sensory data, may be used by analytics engine 206 to determine and an emotional response 220 for output. A change of emotional response 222 may also be determined by analytics engine 206 based on emotional response 220. For example, the change of emotional response 222 may represent a change of emotional response 220 over time, e.g. a derivate of emotional response 220.

In some embodiments, change of emotional response 222 may be used as feedback to analytics engine 206, e.g. to determine the derivative of the change of emotional response.

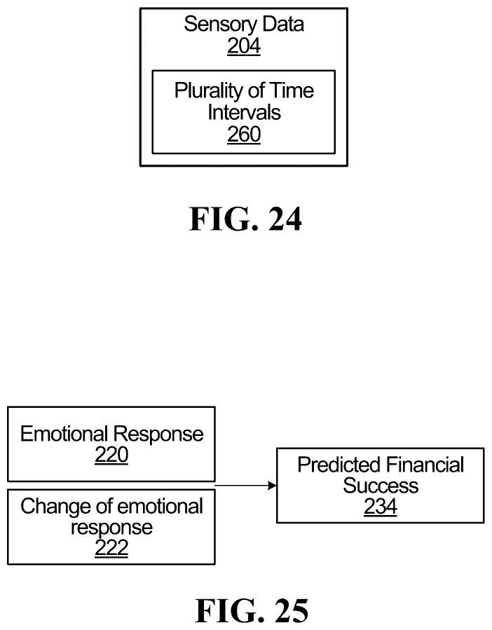

In some embodiments, sensory data 204 may include a plurality of time intervals 260, as depicted in FIG. 24. Change of emotional response 222 over each of plurality of time intervals 260 may be computed by analytics engine 206. Plurality of time intervals 260 may be evenly spaced, monotonic, variable, and/or any other possible type of spacing.

Change of emotional response 222 may be computed over each of the time intervals 260 for each type of sensory data 204, e.g. vision 240, hearing 242, touch 244, taste 246, and/or smell 248. Plurality of time intervals 260 may be different for each type of sensory data 204 and may be based on the respective bandwidth.

In some embodiments, the predicted financial success 234 of external stimuli 202 may be determined, based on at least one of emotional response 220 or change of emotional response 222, as depicted in FIG. 25. For example, analytics engine 206 may measure predicted financial success 234 associated with external stimulus 202 based on at least one of emotional response 220 or change of the emotional response 222, such as by using AI agents 230. As noted above, in some embodiments, AI agents 230 may be trained to emulate a user with a specific disposition.

Computing success metric 224 and/or predicted financial success 234 may depend on the value of the “S” factor with respect to an energy level or emotional energy level classification. In other embodiments, success metric 224 and/or predicted financial success 234 may be computed based on a total weighted value of “S” factors across any number of energy level categories. For example, movies that appeal to a wider population may provide a higher weighted “S” factor across multiple energy levels.

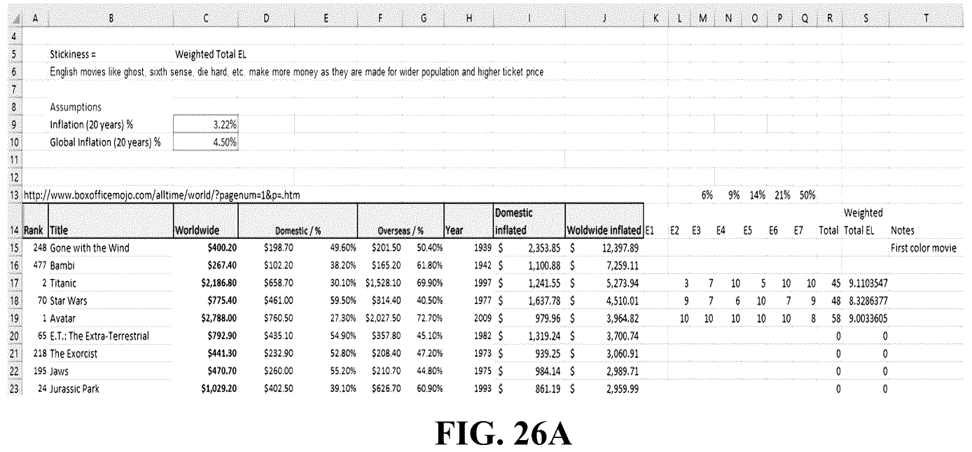

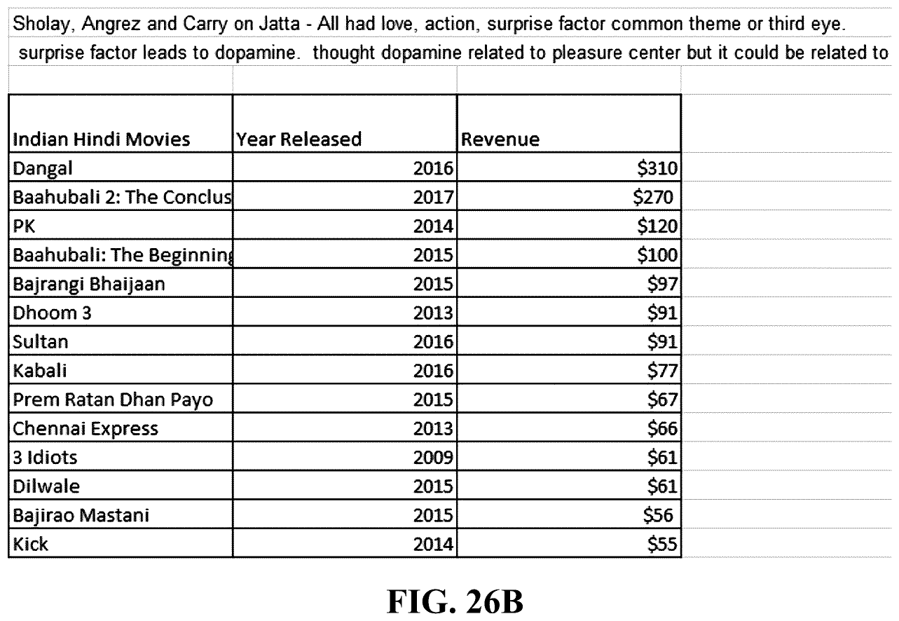

FIGS. 26A-26B depict an example of predicting the financial success of movies. For instance, an “S” factor for one or more energy levels may be computed and weighted across other energy levels to predict the financial success. In some embodiments, analytics engine 206 may use a regression model to assess the predicted revenue, profit, or value. Alternatively, analytics engine 206 may use AI agents 230 and/or models trained to predict the financial success based on external stimuli, sensory data, and/or emotional response.

In some embodiments, several models may be stacked in series following an LLM. For example, the models may include the persona of an experienced, a questioner, an integrator, and a regression model with past sales data.

In some embodiments, a user engagement graph, such as from a video player, may be used to train AI agents 230. For example, the user engagement graph may provide watch time data which may include portions of a video that were skipped or replayed that may be represented by one or more peaks and/or valleys. A peak may indicate a popular or significant moment, whereas a valley may indicate a less interesting portion of the video among viewers.

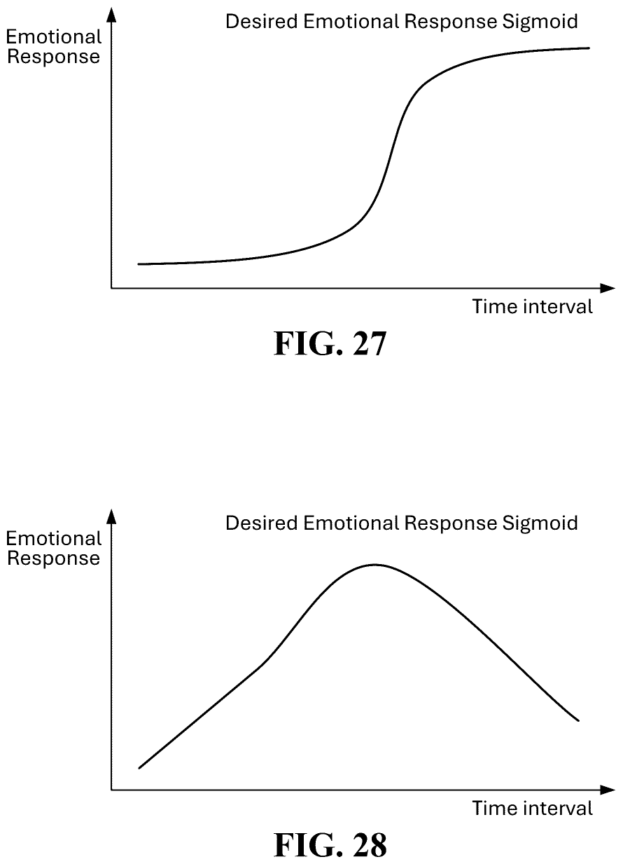

As depicted in FIG. 27, a desirable emotional response may be a sigmoidal curve where the emotional response is at a low value at the beginning of the period of time and rises to a higher value at the conclusion of the period of time. For example, the time interval may be depicted on the x-axis of the curve and the emotional response may be depicted on the y-axis. The desired emotional response may be determined based on human audience 250, external stimulus 202, AI agent 230, disposition profiles 232, and/or another input (e.g. a financial success goal).

As depicted in FIG. 28, an undesirable emotional response may be a curve where the emotional response does not rise to a higher value at the conclusion of the period of time or where the emotional response decreases during the time interval. Other examples may also be possible, such as where the emotional response does not resemble the “S” curve depicted in FIG. 27. The undesirable emotional response may be determined based on human audience 250, external stimulus 202, AI agent 230, disposition profiles 232, and/or another input (e.g. a minimum success metric).

In some embodiments, emotional response 220 may be compared to the desired emotional response. For example, if emotional response 220 is similar to that of the desired emotional response sigmoid, the system may output a higher success metric 224. Alternatively, if emotional response 220 does not mimic or share similarities with the desired response sigmoid, the system may output a lower success metric 224.

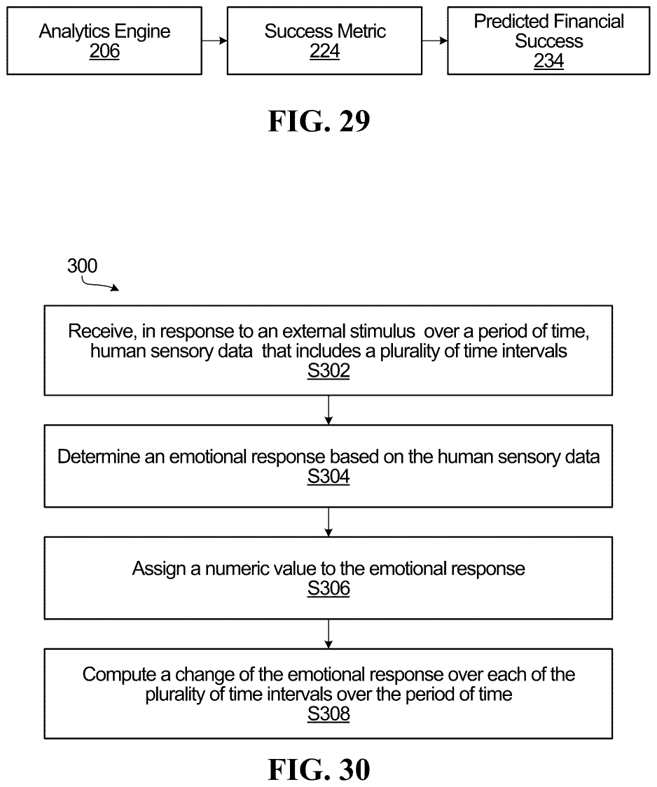

In some embodiments, success metric 224 may be used to measure predicted financial success 234, as depicted in FIG. 29. Analytics engine 206 may determine success metric 224 associated with external stimulus 202 based on the derivative of the change of emotional response over a period of time as well as the change of emotional response 222 over the period of time compared with the desired emotional response.

FIG. 30 depicts a method 300 for determining an emotional response to an external stimulus, such as emotional response 220 and/or change of emotional response 222. Method 300 may be performed by system 200, and in particular using analytics engine 206.

At step S302, human sensory data that includes a plurality of time intervals, in response to an external stimulus over a period of time, is received.

Human sensory data may include vision 240, hearing 242, touch 244, taste 246, and/or smell 248. Sensory data 204 may also include a dopamine concentration and/or serotonin concentration.

Sensory data 204 may be acquired from a human audience 250. Biometric sensors 252 may include measuring a heartbeat, the Galvanic skin response, inflammation using fingerprint expansion, piloerection (e.g. goosebumps), and/or eye-ball dilation. However, other biometric sensors 252 and methods of measurement may be used to acquire human sensory data 204.

In some embodiments, a mapping function, algorithm, or AI model may be used to translate data acquired from biometric sensors 252 to obtain sensory data 204.

In another embodiment, sensory data 204 may be produced by an AI (e.g. using AI agents 230). As used in this sense, produced may also mean simulated, generated, artificially generated and/or fabricated. For example, AI agents 230 may be trained to emulate a user with a specific disposition. Sensory data 204 (e.g. human sensory data) may also be collected from AI agents 230.

Analytics engine 206 may also cause external stimulus 202 to be generated, which may be used to generate sensory data 204. For instance, external stimulus 202 may be generated randomly, based on a saved template, in response to specific input data, and/or using one or more other methods.

In yet another embodiment, AI agents 230 may interpret external stimulus 202 and emulate how a specific user with a specific disposition may respond. For example, AI agents 230 may generate sensory data 204 for a user with a specific disposition, using disposition profiles 232, in response to driving a new car model.

In another example, sensory data 204 may be produced by analytics engine 206 through user engagement analysis, video content analysis and/or natural language processing. The emulated sensory data 204 may be provided as feedback to analytics engine 206.

At step S304, an emotional response is determined based on the human sensory data.

Emotional response 220 may be determined by analytics engine 206 based on sensory data 204.

Determining emotional response 220 may also include a mapping or classification of external stimulus 202 to an energy level. Classification of external stimulus 202 may provide the necessary context for determining emotional response 220 and/or the success. Classification may be performed by analytics engine 206 using an LLM and/or AI agents 230 and/or disposition profiles 232. Mapping may also be performed manually, in some embodiments.

Analytics engine 206 may compute emotional response 220 and/or the “S” factor for a user, based on the specific disposition. For example, for a disposition profile of user(s) with a weaker response to human love, analytics engine 206 may compute a lower “S” factor for a romance movie scene.

At step S306, a numeric value to the emotional response is assigned.

A numerical value may be assigned to emotional response 220. For instance, the “S” factor over time for a user's response to external stimulus 202, based on sensory data 204, may represent or be used as a proxy for emotional response 220.

At step S308, a change of the emotional response over each of the plurality of time intervals over the period of time is computed.

Change of emotional response 222 over each of plurality of time intervals 260 may be computed by analytics engine 206. Plurality of time intervals 260 may be evenly spaced, variable, or any other possible type of spacing.

Change of emotional response 222 may be computed over each of the time intervals 260 for each type of sensory data 204, e.g. vision 240, hearing 242, touch 244, taste 246, and/or smell 248.

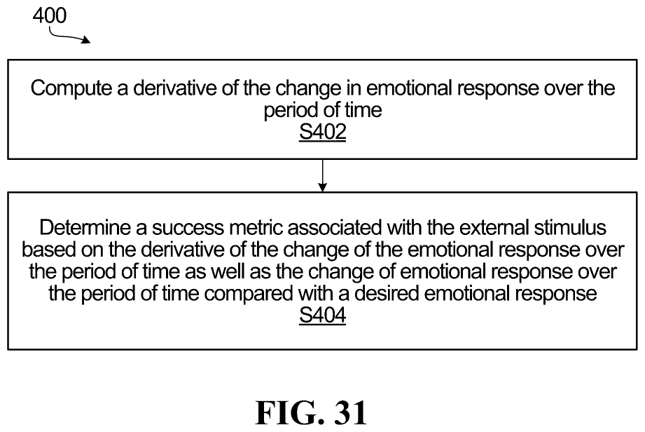

FIG. 31 depicts a method 400 for determining the success based on the emotional response. Method 400 may be performed by system 200, and in particular using analytics engine 206. Method 400 may be performed in combination with method 300.

At step S402, a derivative of the change in emotional response over the period of time is computed.

In one embodiment, output 208 may provide feedback to analytics engine 206 to determine the derivative of the change of emotional response. For example, the derivative of the “S” factor with respect to time may be computed to represent the derivative of change of emotional response 222.

In another embodiment, output 208 may provide feedback to analytics engine 206 to determine a second derivative of the change of emotional response.

At step S404, a success metric associated with the external stimulus is determined based on the derivative of the change of the emotional response over the period of time as well as the change of emotional response over the period of time compared with a desired emotional response.

Success metric 224 may be a prediction of financial success, such as the predicted revenue, gross profit, or net income. For example, external stimulus 202 may be a form of media and success metric 224 may be determined by analytics engine 206, which may describe the predicted success of the media (e.g. financial success). In other embodiments, success metric 224 may describe other aspects of external stimulus 202, such as a ranking or rating, including with respect to other external stimuli.

Success metric 224 may alternatively or additionally be an engagement level or loyalty change. In another example, external stimulus 202 may be an experience 216, e.g. the process of driving a new car model. Success metric 224 may be determined by analytics engine 206, which may represent the predicted increase in loyalty of a user after buying the new car. In another example, external stimulus 202 may be a specific experience associated with a new car model, such as sitting in the driver's seat, opening the trunk, or backing up out of a driveway.

Computing success metric 224 and/or predicted financial success 234 may depend on the value of the “S” factor with respect to an energy level. In other embodiments, success metric 224 and/or predicted financial success 234 may be computed based on a total weighted value of “S” factors across any number of energy level categories. For example, movies that appeal to a wider population may provide a higher weighted “S” factor across multiple energy levels.

In one embodiment, the derivative of the change of the emotional response over the period of time may be determined based on the “S” factor. For example, the derivative of the “S” factor with respect to time may be computed and used to determine the success.

The desirable emotional response may be a sigmoidal curve, where the emotional response is at a low value at the beginning of the period of time and rises to a higher value at the conclusion of the period of time. The desired emotional response may be determined based on human audience 250, external stimulus 202, AI agent 230, disposition profiles 232, and/or another input (e.g. a financial success goal).

An undesirable emotional response may be a curve where the emotional response does not rise to a higher value at the conclusion of the period of time or where the emotional response decreases during the time interval. The undesirable emotional response may be determined based on human audience 250, external stimulus 202, AI agent 230, disposition profiles 232, and/or another input (e.g. a minimum success metric)

Emotional response 220 may be compared to the desired emotional response. For example, if emotional response 220 is similar to that of the desired emotional response sigmoid, the system may output a higher success metric 224. Alternatively, if emotional response 220 does not mimic the desired response sigmoid, the system may output a lower success metric 224.

The desired emotional response could be specific to an energy level, user profile, or other factor. For example, the desired emotional response used for comparison may be different for a comedy versus an action movie. Similarly, the desired emotional response may depend on user demographics and objectives.

Optionally, output 208 may further include a recommendation 226. Analytics engine 206 may optionally use recommendation 226 and/or success metric 224 to adapt external stimulus 202 (e.g. change the appearance of a new car model). For instance, analytics engine 206 may modify, revise, delete from, add to, or re-generate external stimulus 202 (e.g. change the colour of the new car model). Adaption and/or re-generation of external stimulus 202 may be performed by analytics engine 206.

Alternatively, recommendation 226 may provide instructions to analytics engine 206 to adapt one or more aspects of sensory data 204 that will result in a desired success metric and generate an external stimulus that produces said sensory data. Analytics engine 206 may output a new success metric based on the adapted external stimulus 202 and generated sensory data 204.

Analytics engine 206 may generate recommendation 226 based on success metric 224 associated with external stimulus 202, which may be used to adapt external stimulus 202. Analytics engine 206 may continue analyzing external stimulus 202 and/or sensory data 204 any number of times, which may depend on recommendation 226 and/or success metric 224.

### Example Movie Analytics System

The following example of a movie analytics system may be included or be performed by the previously described system 100 and/or system 200.

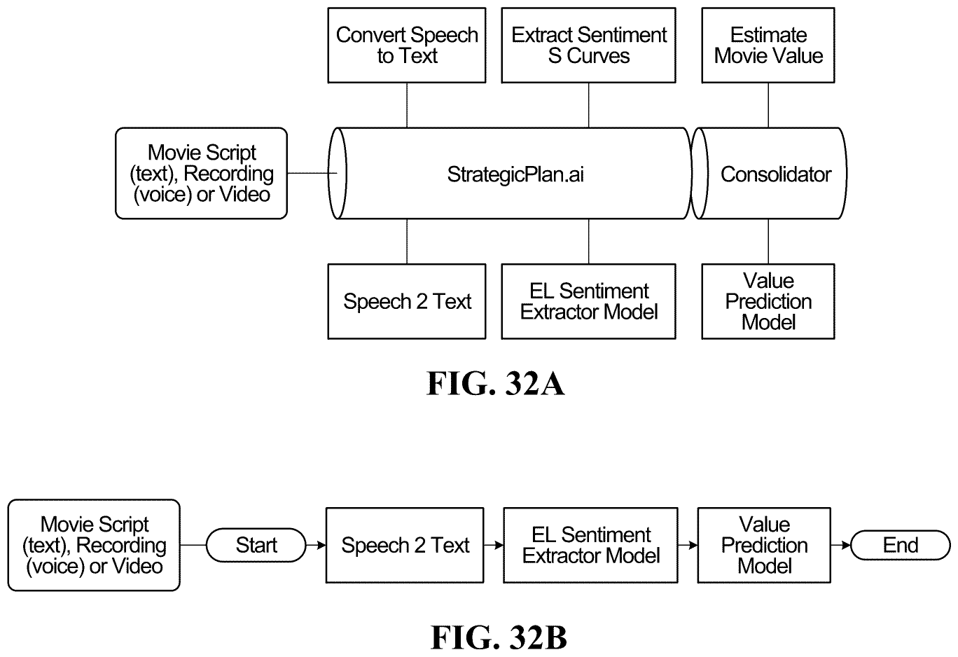

As depicted in FIGS. 32A-B, a movie analytics system may be implemented using analytics system 200 to predict the movie value. For example, a movie script, recording, or video (external stimulus 202) may be received by a strategic plan AI agent, which may interface with other modules as previously described. The strategic plan AI agent may include or host other AI agents programmed to perform other specialized tasks.

The movie recording or video may be converted to text using a speech to text translation AI model or agent. Sentiment “S” curves (e.g. surprise factors) may be extracted based on the text conversion. In some embodiments, the sentiment curves may be computed by AI agents 230 trained to emulate a specific disposition. AI agents may generate or produce sensory data 204 for a specific disposition in computing the sentiment curves.

Moreover, the strategic plan AI agent may compute emotional response 220 and/or change in emotional response based on external stimulus 202.

A consolidator may receive emotional response 220 and/or the sentiment curves and compute an estimated movie value using a value prediction model. For instance, predicted financial success 234 of external stimuli 202 may be determined, based on at least one of emotional response 220 or change of emotional response 222.

Success metric 224 and/or predicted financial success 234 may be computed based on a total weighted value of “S” factors across any number of energy levels. For example, movies that appeal to a wider population may provide a higher weighted “S” factor across multiple energy levels and a higher movie value estimation.

Other modifications and embodiments are possible within the example movie analytics engine. As well, the description above is not intended to be limiting to the embodiments and embodiments described herein.

In some further embodiments of the methods and systems described herein, the methods and systems may also be used to generate new content based on the sigmoidal curve. The new content may include text, image, video and/or other media. The desired emotional response of the new content may begin at a low value at the beginning of a period of time and may rise to a higher value at the conclusion of the period of time, as described above. This desired emotional response may be used to create new content with a higher stickiness factor for audiences.

### Example Computing Device

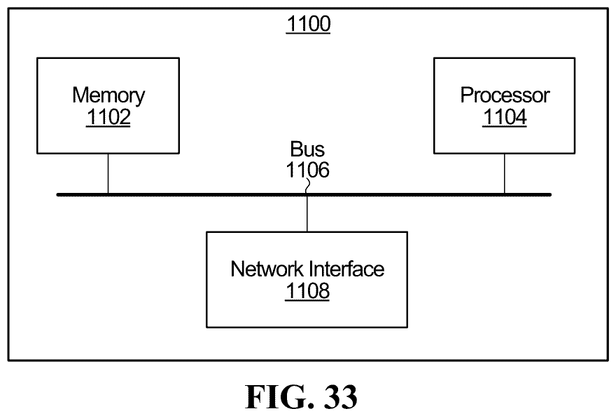

FIG. 33 is a schematic diagram of a computing device 1100 configured to implement the components of system 100 and/or system 200, according to some embodiments. Computing device 1100 includes a memory 1102, a processor 1104 and a bus 1106. Computing device 1100 may also include a network interface 1108. A communication connection is implemented between the memory 1102, the processor 1104, and the network interface 1108 by using the bus 1106.

The processor 1106 and the network interface 1108 are configured to perform, when the program or computer-executable instructions stored in the memory 1102 is/are executed by the processor 1104, steps of method 300 and/or method 400. The processor 1104 and the network interface 1108 may also be configured to perform, when the program or computer-executable instructions stored in the memory 1102 is/are executed by the processor 1104, any other processes or modules discussed with respect to system 100, system 200, system 800, analytics engine 104, analytics engine 104′ and/or analytics engine 206.

The memory 1102 may be a read-only memory (Read Only Memory, ROM), a static storage device, a dynamic storage device, or a random access memory (Random Access Memory, RAM). The memory 1102 may store a program or computer-executable instructions. The memory 1102 may be a non-transitory memory.

The processor 1104 may be a general central processing unit (Central Processing Unit, CPU), a microprocessor, an application-specific integrated circuit (Application Specific Integrated Circuit, ASIC), a graphics processing unit (graphics processing unit, GPU), or one or more integrated circuits.

In addition, the processor 1104 may be an integrated circuit chip with a signal processing capability. In an embodiment process, steps of method 300 and/or method 400 may be performed by an integrated logical circuit in a form of hardware or by an instruction in a form of software in the processor 1104. In addition, the processor 1102 may be a general purpose processor, a digital signal processor (Digital Signal Processor, DSP), an ASIC, a field programmable gate array (Field Programmable Gate Array, FPGA) or another programmable logic device, a discrete gate or a transistor logic device, or a discrete hardware assembly.

The processor 1102 may implement or execute the methods, steps, and logical block diagrams that are disclosed in the example embodiments. The general-purpose processor may be a microprocessor, or the processor may be any conventional processor or the like. The steps of the methods disclosed herein may be directly performed by a hardware decoding processor, or may be performed by using a combination of hardware in the decoding processor and a software module. The software module may be located in a mature storage medium in the art, such as a random access memory, a flash memory, a read-only memory, a programmable read-only memory, an electrically erasable programmable memory, or a register. The storage medium may located in the memory 1102. The processor 1104 may read information from the memory 1104 and complete, by using hardware in the processor 1104, the steps of method 300 and/or method 400.

The network interface 1108 may implement communication between computing device 1100 and one or more other devices and/or computing devices over a communications network, such as by using a transceiver apparatus, for example, including but not limited to a transceiver. For example, components of system 100 and/or system 200 may be configured to communicate with one another over a communications network. In a particular example, user device 102 and analytics engine 104 may communicate with one another using their own respective network interfaces.

The bus 1106 may include a path and/or communication channel that transfers information between all the components of the computing device 1100.

It should be noted that, although only the memory, the processor, and the communications interface are shown in the computing device in FIG. 33, in a specific embodiment process, a person skilled in the art should understand that system 100 and/or system 200, as well as analytics engine 104, analytics engine 104′, and/or analytics engine 206, may further include other components that are necessary for embodiment, such as one or more additional computing devices, servers, networks, memories, processors, etc.

In addition, based on specific needs, a person skilled in the art should understand that the components of these systems may further include hardware components that implement other additional functions. In addition, a person skilled in the art should understand that system 100 and/or system 200 may include only a component required for implementing the embodiments of the present invention, without a need to include all the components shown in FIG. 33.

In the described methods, the boxes may represent events, steps, functions, processes, modules, state-based operations, etc. While some of the above examples have been described as occurring in a particular order, it will be appreciated by persons skilled in the art that some of the steps or processes may be performed in a different order provided that the result of the changed order of any given step will not prevent or impair the occurrence of subsequent steps.

Furthermore, some of the messages or steps described above may be removed or combined in other embodiments, and some of the messages or steps described above may be separated into a number of sub-messages or sub-steps in other embodiments. Even further, some or all of the steps may be repeated, as necessary. Elements described as methods or steps similarly apply to systems or subcomponents, and vice-versa. Reference to such words as “sending” or “receiving” could be interchanged depending on the perspective of the particular device, module or logical element.

While some example embodiments have been described, at least in part, in terms of methods, a person of ordinary skill in the art will understand that some example embodiments are also directed to the various components for performing at least some of the aspects and features of the described processes, be it by way of hardware components, software or any combination of the two, or in any other manner.

Moreover, some example embodiments are also directed to a pre-recorded storage device or other similar computer-readable medium including program instructions stored thereon for performing the processes described herein. The computer-readable medium includes any non-transient storage medium, such as RAM, ROM, flash memory, compact discs, USB sticks, DVDs, HD-DVDs, or any other such computer-readable memory devices.

It will be understood that the devices described herein include one or more processors and associated memory. The memory may include one or more application program, modules, or other programming constructs containing computer-executable instructions that, when executed by the one or more processors, implement the methods or processes described herein.

As used herein, an artificial intelligence agent may include at least one AI model, such as a large language model or multi-model large language model. An AI agent may also include one or more other models or tools to allow artificial intelligence agents to perform one or more tasks. Furthermore, as used herein, the term artificial intelligence agent may also refer to a single artificial intelligence agent or one or more artificial intelligence agents, such as a group of artificial intelligence agents.

The various embodiments presented are merely examples and are no way meant to limit the scope of example embodiments. Variations of the innovations described will be apparent to persons of ordinary skill in the art, such variations being within the intended scope of the example embodiments. In particular, features from the described embodiments may be selected to create alternative embodiments comprised of a sub-combination of features which may not be explicitly described.

In addition, features from one or more of the embodiments may be selected and combined to create alternative embodiments comprised of a combination of features which may not be explicitly described. Features suitable for such combinations and sub-combinations would be readily apparent to persons skilled in the art upon review of the example embodiments. The subject matter described herein intends to cover all suitable changes in technology.

Certain adaptions and modifications of the described embodiments can be made. Therefore, the described embodiments are considered to be illustrative.

The computer described may be a computing device, such as a mobile device, a personal computer, a server, an embedded system or some other device with computing capabilities.

1. A computer-implemented method, the method comprising:
receiving, in response to an external stimulus over a period of time, human sensory data that comprises a plurality of time intervals;
determining an emotional response based on the human sensory data;
assigning a numeric value to the emotional response; and
computing a change of the emotional response over each of the plurality of time intervals over the period of time.

2. The computer-implemented method of claim 1, wherein the external stimulus is a form of media, comprising at least one of an advertisement, a video, a film, text, an image, or audio, the method further comprising predicting the financial success of the media based on at least one of the emotional response or change of the emotional response.

3. The computer-implemented method of claim 1, wherein the human sensory data comprises data describing at least one of vision, hearing, touch, taste or smell.

4. The computer-implemented method of claim 1, wherein the human sensory data is collected by biometric sensors or manual observation.

5. The computer-implemented method of claim 2, wherein a desired emotional response is a sigmoidal curve where the emotional response is at a low value at the beginning of the period of time and rises to a higher value at the conclusion of the period of time.

6. The computer-implemented method of claim 1, wherein the human sensory data is produced by an artificial intelligence agent.

7. The computer-implemented method of claim 6, wherein the artificial intelligence agent is trained to emulate a user with a specific disposition.

8. The method of claim 1, the method further comprising:
computing a derivative of the change in emotional response over the period of time; and
determining a success metric associated with the external stimulus based on the derivative of the change of the emotional response over the period of time as well as the change of emotional response over the period of time compared with a desired emotional response.

9. The computer-implemented method of claim 1, the method further comprising generating a recommendation to adapt the external stimulus based on a success metric associated with the external stimulus.

10. A system, the system comprising:
A user interface component; and
A server component configured to:
receive in response to an external stimulus over a period of time, human sensory data that comprises a plurality of time intervals;
determine an emotional response based on the human sensory data;
assign a numeric value to the emotional response;
compute a change of the emotional response over each time of the plurality of time intervals over the period of time.

11. The system of claim 10, wherein the external stimulus is a form of media, comprising at least one of an advertisement, a video a film, text, an image, or audio, the system further configured to predict financial success of the media based on at least one of the emotional response or change of the emotional response.

12. The system of claim 10, wherein the human sensory data comprises data describing at least one of vision, hearing, touch, taste or smell.

13. The system of claim 10, wherein the human sensory data is collected by biometric sensors or manual observation.

14. The system of claim 11, wherein a desired emotional response is a sigmoidal curve where the emotional response is at a low value at the beginning of the period of time and rises to a higher value at the conclusion of the period of time.

15. The system of claim 10, wherein the human sensory data is produced by an artificial intelligence agent.

16. The system of claim 15, wherein the artificial intelligence agent is trained to emulate a user with a specific disposition.

17. The system of claim 10, the server component further configured to:
compute a derivative of the change in emotional response over the period of time; and
determine a success metric associated with the external stimulus based on the derivative of the change of the emotional response over the period of time as well as the change of emotional response over the period of time compared with a desired emotional response.

18. The system of claim 11, wherein the server is further configured to generate a recommendation to the external stimulus based on a success metric associated with the external stimulus.

19. One or more non-transitory computer readable media storing executable instructions thereon that, when executed by at least one computer, cause the at least one computer to perform a method comprising:
receiving, in response to an external stimulus over a period of time, human sensory data that comprises a plurality of time intervals;
determining an emotional response based on the human sensory data;
assigning a numeric value to the emotional response; and
computing a change of the emotional response over each of the plurality of time intervals over the period of time.

20. The one or more non-transitory computer readable media of claim 19, wherein the external stimulus is a form of media, comprising at least one of an advertisement, a video a film, text, an image, or audio, the method further comprising predicting the financial success of the media based on at least one of the emotional response or change of the emotional response.
